(19) United States
(12) Patent Application Publication
(10) Pub. No.: US 2025/0186494 A1
(43) Pub. Date: Jun. 12, 2025

(54) CAR-T THERAPIES TARGETED VIA COVALENTLY BONDED ADAPTERS

(71) Applicant: THE SCRIPPS RESEARCH INSTITUTE, La Jolla, CA (US)

(72) Inventors: Richard A. LERNER, La Jolla, CA (US); Jia XIE, Davie, FL (US); Alexey STEPANOV, San Diego, CA (US)

(21) Appl. No.: 18/837,487

(22) PCT Filed: Feb. 7, 2023
(86) PCT No.: PCT/US2023/062141

§ 371 Date: Aug. 9, 2024

(51) Int. Cl. A61K 35/17; A61K 40/11; A61K 40/31; A61K 40/42; C07K 14/725; C07K 16/44; C12N 5/0783

(52) U.S. Cl. CPC A61K 35/17; A61K 40/11; A61K 40/31; A61K 40/4205; A61K 40/4276; C07K 14/7051; C07K 16/44; C12N 5/0636; A61K 2239/13; A61K 2239/21; A61K 2239/24; A61K 2239/49; A61K 2239/58; C07K 2317/24; C07K 2317/622; C07K 2319/03

(57) ABSTRACT

The invention provides chimeric antigen receptor T (CAR-T) cell compositions for targeting tumor cells. The compositions contain (a) a CAR-T cell having in the extracellular domain of its CAR a catalytic antibody (e.g., a scFv molecule derived from catalytic antibody 38C2), and (b) an adapter compound containing a substrate moiety of the catalytic antibody that is linked to a targeting moiety that specifically recognizes a surface molecule of a target tumor cell. The compositions allow formation of a covalent bond between the catalytic antibody in the CAR and the targeting moiety. The targeting moieties employed in the compositions can be obtained via screening DNA-encoded compound library for specific binding to the target tumor surface molecules. Also provided in the invention are therapeutic methods of using the CAR-T cell compositions of the invention to in the treatment of various tumors of interest.

### CROSS-REFERENCE TO RELATED APPLICATIONS

The subject patent application claims the benefit of priority to U.S. Provisional Patent Application No. 63/308,702 (filed Feb. 10, 2022; now pending). The full disclosure of the priority application is incorporated herein by reference in its entirety and for all purposes.

### BACKGROUND OF THE INVENTION

Chimeric antigen receptor T cell (CAR-T) therapy has gained much attention in recent years owing to its well-recognized success in the treatment of chronic lymphocytic leukemia (CLL). Very often CAR-T therapy can deliver significant remission rates, and it has the potential to become part of standard-of-care treatment for CLL. However, current CAR-T therapy is still hampered by a variety of drawbacks. One of the most troubling issues concerns the frequent induction of Cytokine Release Syndrome (CRS) resulting from overactivation of the T cells and on-target off-tumor effects. CAR-T therapy involves the injection of living cells with the capacity for replication. Unlike the usual side effects with pharmaceuticals that can be ameliorated by withholding drug, or reducing dosage, it is much more difficult to regulate once it is initiated. A few recent investigations have attempted to use small molecule activators or antibody adapters to harness the activity of specially engineered CAR-T cells in a controlled manner. Some of these studies have shown encouraging results.

Nonetheless, there is a need in the art for CAR-T therapies for more effectively treating various malignancies. The present invention is directed to this and other unmet needs in the art.

### SUMMARY OF THE INVENTION

In one aspect, the invention provides chimeric antigen receptor T (CAR-T) cell compositions for targeting tumors. The compositions contain (a) a CAR-T cell having in the extracellular domain of its CAR a catalytic antibody, and (b) an adapter compound containing a substrate moiety of the catalytic antibody that is linked (fused/conjugated) to a targeting moiety that specifically recognizes a surface molecule of a tumor cell. In some embodiments, the catalytic antibody is a single chain fragment variable (scFv) molecule. In some embodiments, the catalytic antibody is capable of catalyzing formation of a covalent bond between the antibody and the substrate moiety. In some of these embodiments, the covalent bond is formed between the substrate moiety and a reactive amino acid residue of the catalytic antibody. In some embodiments, the employed catalytic antibody is catalytic antibody 38C2 or variant thereof. In some of these embodiments, the employed catalytic antibody is humanized antibody 38C2 (h38C2). In some of these compositions, the employed antibody is a scFv of h38C2. Some compositions of the invention contain a scFv 38C2 that has the light chain variable region (VL) sequence linked at its C-terminus by a Gly-Ser linker to the N-terminus of the heavy chain variable region (VH) sequence.

In some embodiments, the employed substrate moiety contains a small molecule. In some of these embodiments, the substrate moiety contains 1,3-diketone or β-lactam. In some other embodiments, the targeting moiety is identified via screening a DNA-encoded compound library for specific binding to the tumor cell surface molecule. In some embodiments of the invention, the targeting moiety targets PSMA. In some of these embodiments, the targeting moiety contains compound DEL507, DEL147, DEL19, DEL 164, DEL79, DEL79/2, DEL79/1, DEL1346/6 or DEL 1346/10 as shown in FIG. 6. In some other embodiments of the invention, the targeting moiety targets HER2. In some of these embodiments, the targeting moiety contains compound DEL675/1, DEL675/3, DEL675/5, DEL675/9 or DEL675/11 as shown in FIG. 7.

In a related aspect, the invention provides methods for targeting a CAR-T cell to a tumor cell present in a population of heterogeneous cells. These methods involve contacting the population of cells with a CAR-T composition that contains (a) a CAR-T cell having in the extracellular domain of its CAR a catalytic antibody, and (b) an adapter compound containing a substrate moiety of the catalytic antibody that is linked to a targeting moiety that specifically recognizes a surface molecule of the target tumor cell. In some of these methods, the catalytic antibody catalyzes formation of a covalent bond between the antibody and the substrate moiety. In some embodiments, the covalent bond is formed between the substrate moiety and a reactive amino acid residue of the catalytic antibody. In some methods, the employed catalytic antibody is catalytic antibody 38C2 or variant thereof. Some of these methods employ is humanized antibody 38C2 or an antibody fragment thereof, e.g., a scFv of h38C2. In some methods, the employed substrate moiety contains a small molecule. For example, the substrate moiety can contain 1,3-diketone or β-lactam. Some of the methods can additionally entail identifying a suitable targeting moiety via screening a DNA-encoded compound library for specific binding to the tumor cell surface molecule. In some embodiments, the tumor cell to be targeted is a breast cancer cell or a prostate cancer cell.

In another aspect, the invention provides methods for treating a cancer in a subject. These methods entail administering to the subject a pharmaceutical composition that contains (a) a CAR-T cell having in the extracellular domain of its CAR a catalytic antibody, and (b) an adapter compound containing a substrate moiety of the catalytic antibody that is linked to a targeting moiety that specifically recognizes a surface molecule of the cancer. In some of these methods, the employed catalytic antibody catalyzes formation of a covalent bond between the antibody and the substrate moiety. In some of these embodiments, the covalent bond is formed between the substrate moiety and a reactive amino acid residue of the catalytic antibody. In some embodiments, the employed catalytic antibody is catalytic antibody 38C2 or variant thereof. In some of these embodiments, the catalytic antibody can be humanized antibody 38C2 or an antibody fragment thereof. For example, some of the methods can use a scFv of h38C2. In some embodiments, the employed substrate moiety contains a small molecule. For example, the substrate moiety can contain 1,3-diketone or β-lactam. In some other embodiments, the methods can additionally include identifying the targeting moiety via screening a DNA-encoded compound library for specific binding to the tumor cell surface molecule. In some methods, the subject to be treated is afflicted with breast cancer or prostate cancer.

A further understanding of the nature and advantages of the present invention may be realized by reference to the remaining portions of the specification and claims.

### DESCRIPTION OF THE DRAWINGS

FIG. 1. Design and characterization of a covalent CAR-T cell and folate receptor adapter molecules. (A) Demonstration that folic acid receptor is expressed on KB cells and absent on control A549 cells. (B) Structure of fluorescein-folate, fluorescein-diketone and folate-diketone. (C) Covalent labeling of aldolase antibody 38C2 with a diketone conjugate. (D) Schematic of covalent CAR construct design, where V_{L} and VH=variable domains of the 38C2 catalytic antibody, GS-Gly-Ser linker, wl=Whitlow linker, TM=transmembrane domain, 4-1BB=cytoplasmic activation domain from CD137, CD3ζ=the cytoplasmic activation domain of CD3 zeta. (E) Comparison of the expression efficacy and catalytic activity of the T cells transduced by designed covalent CARs. Cells were labeled with anti-IgG4 antibodies (CAR detection, left panel) and fluorescein-diketone (right panel). (F) Comparison of the cytotoxicity and non-specific lysis of the designed CovCARs v1-v4 toward KB cells upon addition of 25 nM folate-diketone or no adapter. The ratio of CovCAR-T cells to KB cells was 10 to 1 (n=4). (G) Covalent modification of the CovCAR-T cell surface by the fluorescein-diketone, spacer domain of CAR was labeled by anti-IgG4 PE antibodies.

FIG. 2. In vitro and in vivo activity of CovCAR-T cells and folate-diketone in KB xenograft model. (A) Effect of folate-diketone concentrations on FR-positive KB and FR-negative A549 cell lysis by CovCAR-T cells (left). Impact of effector: target cell ratio on CovCAR-T cell lysis of KB and A549 cells in presence of folate-diketone concentration 12.5 nM (right) (n=4). (B) CovCAR-T cells release IL-2 and IFNg cytokines in presence of KB cells and folate-diketone in a dose-dependent manner (n=4). (C) NSG mice were implanted s.c. with 2 million KB cells and 10 million CovCAR-T cells. Mice were then given daily i.p. injections of either PBS or folate-diketone (75 nmole/kg). The mice received a total of one dose of CovCAR-T cells and 15 doses of folate-diketone or control during the next 15 days. (D) Survival plots for animals treated with PBS or folate-diketone. Overall survival curves were plotted using the Kaplan-Meier method and compared using the log-rank (Mantel-Cox) test. Statistical significance: * p<0.0001. (E) Representative IVIS images of the mice from folate-diketone and PBS treated groups. (F) Quantified tumor burden (as average radiance from luciferase activity from each mouse) from (E) for the 7-28 day period. (G and H) Tumor growth kinetics of tumor-bearing mice from folate-diketone and PBS treated groups. n=8 mice per group. All data represent mean+SD. The p values were determined by multiple t test. Statistical significance: * p<0.05, ** p<0.01, *** p<0.001.

FIG. 3. Design and characterization of a covalent CAR-T cell and PSMA adapter molecules. (A) Chemical structures of the synthesized PSMA-specific diketone conjugates. All bispecific molecules have the same PSMA and diketone CovCAR recognition domains and vary only in the linker length (DUPA-1/5-diketone). (B) Demonstration that PSMA is expressed on prostate cancer PC-3 cells transduced with PSMA (left) and LNCaP (right) cells and absent on control PC-3 cells. (C) Orthogonal or dual-targeted elimination of the heterogeneous prostate cancer cells by the CovCAR-T cells controlled by the small-drug conjugates specificity and concentration. A mixture of the PC-3, PC-3 PSMA and PC-3 FR cells was incubated with CovCAR-T cells with different folate-diketone/DUPA-3-diketone conjugates compositions and analyzed by flow cytometry. The heat map graph represents the percentage of live cells of each population normalized to negative control (no CAR-T cells added) (D) The confocal microscopy of CovCAR-Ts labeled by CFSE and PC-3 PSMA labeled by CellTrace FarRed after one hour of co-incubation in presence of 1 nM DUPA-3-diketone or PBS. (E) Effect of PSMA-diketone conjugates concentration on cytokine release and cancer cell lysis by the CovCAR-T cells in vitro. The PC-3 PSMA were incubated in the presence of CovCAR-T cells plus different concentrations of PSMA-specific diketone (DUPA-1/5-diketone) or folate-diketone (neg. control) conjugates prior to analysis of tumor cell lysis, IL-2 and IFNg cytokines release (effector: tumor cell ratio 2:1).

FIG. 4. In vivo activity of CovCAR-T cells and DUPA-3-diketone, DUPA-4-diketone and DUPA-5-diketone conjugates in prostate cancer xenograft model. (A) NSG mice were implanted s.c. with 1 million PC3-PSMA cells. On day 8 after tumor inoculation 20×10⁶ CovCAR-T cells with transduction efficacy of 60-75% were infused intravenously into animals from the all experimental groups. Mice were then intravenously injected with either PBS or increasing amounts of the DUPA-3-diketone, DUPA-4-diketone and DUPA-5-diketone conjugates (i.e., 5 nmol/kg on day 8, 50 nmol/kg on day 10, 500 nmol/kg on day 12). After first course of therapy mice were rested for ten days, then the cycle was repeated. (B) Representative IVIS images of the mice from placebo, DUPA-3-diketone, DUPA-4-diketone and DUPA-5-diketone treated groups. (C) Quantified tumor burden (as average radiance from luciferase activity per mouse) from (B) for 6-57 day period. (D) Tumor growth kinetics of mice from control and treated groups. (E) Survival plots for animals from experimental and control groups. Overall survival curves were plotted using the Kaplan-Meier method and compared using the log-rank (Mantel-Cox) test. Statistical significance: ** p<0.01. All data represent mean+SD.

FIG. 5. Pharmacological control of cytokine release and CovCAR-T propagation following DUPA-3-diketone adapter administration in vivo. (A) NSG mice were implanted s.c. with 1 million PC3-PSMA cells. On day 12 after tumor inoculation animals in all experimental groups received an intravenous infusion of 10×10⁶ CovCAR-T cells expressing luciferase. Mice were then injected intravenously with either PBS or increasing amounts of the DUPA-3-diketone conjugate (i.e., 5 nmol/kg on day 12, 50 nmol/kg on day 14, 500 nmol/kg on day 16, 5 nmol/kg on day 29, 50 nmol/kg on day 31, 500 nmol/kg on day 33). (B) Representative IVIS images of the CovCAR-T expansion in mice from placebo and DUPA-3-diketone treated groups. (C) Peripheral blood IFNg and Perforin concentrations on days 16, 31 and 34. (D) Quantified CovCAR-T expansion (as average radiance from luciferase activity per mouse) from (B) for 4-38 day period. Statistical significance: * p<0.05, *** p<0.001 All data represent mean+SD.

FIG. 6. In vitro and in vivo activity of CovCAR-T cells and folate-diketone in KB xenograft model. (A) Effect of folate-diketone concentrations on FR-positive KB and FR-negative A549 cell lysis by CovCAR-T cells (left). Impact of effector: target cell ratio on CovCAR-T cell lysis of KB and A549 cells in presence of folate-diketone concentration 12.5 nM (right) (n=4). (B) CovCAR-T cells release IL-2 and IFNg cytokines in presence of KB cells and folate-diketone in a dose-dependent manner (n=4). (C) NSG mice were implanted s.c. with 2 million KB cells and 10 million CovCAR-T cells. Mice were then given daily i.p. injections of either PBS or folate-diketone (75 nmole/kg). The mice received a total of one dose of CovCAR-T cells and 15 doses of folate-diketone or control during the next 15 days. (D) Survival plots for animals treated with PBS or folate-diketone. Overall survival curves were plotted using the Kaplan-Meier method and compared using the log-rank (Mantel-Cox) test. Statistical significance: * p<0.0001. (E) Representative IVIS images of the mice from folate-diketone and PBS treated groups. (F) Quantified tumor burden (as average radiance from luciferase activity from each mouse) from (E) for the 7-28 day period. (G) Tumor growth kinetics of tumor-bearing mice from folate-diketone and PBS treated groups. n=8 mice per group. All data represent mean+SD. The p values were determined by multiple t test. Statistical significance: * p<0.05, ** p<0.01, *** p<0.001.

FIG. 7. Selection and characterization of HER2-specific adapter molecules from DEL for covalent CAR-T cell therapy. (A) Fingerprint of HER2 selection plotted in 3D representation. Identified structures of the HER2 binders selected from the Wu-Xi-Lib3 DEL library. (B) Effect of HER2-specific DEL-diketone conjugates on SK-BR-3 lysis by the CovCAR-T cells (C) Chemical structures of the synthesized HER2-specific DEL675/1-4-diketone and DEL675/9-4-diketone conjugates. (D) Effect of DEL675/1-4-diketone and DEL675/9-4-diketone conjugate concentrations on SK-BR-3 lysis by CovCAR-T cells in vitro. SK-BR-3 cells were incubated in the presence of CovCAR-T cells plus different concentrations of HER2-specific DEL675/1-4-diketone and DEL675/9-4-diketone conjugates prior to analysis of tumor cell lysis (effector: tumor cell ratio 2:1). (E) Effect of DEL675/1-4-diketone and DEL675/9-4-diketone conjugate concentrations on cytokine release by the CovCAR-T cells in vitro. The SK-BR-3 cells were incubated in the presence of CovCAR-T cells or untransduced T cells plus different concentrations of HER2-specific DEL675/1-4-diketone and DEL675/9-4-diketone conjugates prior to analysis of IL-2 and IFNg cytokine release (effector: tumor cell ratio 5:1).

FIG. 8. Effect of folate-diketone concentration on cell lysis and cytokine release of acute monocytic leukemia and T cell leukemia by the CovCAR-T cells in vitro. (A) Characterization of THP-1, THP-1 FRa and THP-1 FRb cell expression of the folate receptors. (B) The THP-1, THP-1 FRa and THP-1 FRb cells were incubated in the presence of CovCAR-T cells plus different concentrations of folate-diketone prior to analysis of tumor cell lysis (effector: tumor cell ratio 5:1). (C and D) CovCAR-T cells release IL-2 and IFNg cytokines in the presence of acute monocytic leukemia cells expressing the folate receptor and folate-diketone in a dose-dependent manner (effector: tumor cell ratio 5:1). (E) Impact of effector: target cell ratio on CovCAR-T cell lysis of Jurkat and Jurkat FRb cells in presence of folate-diketone concentration 5 nM. (F and G) CovCAR-T cells release IL-2 and IFNg cytokines in presence of folate receptor expressing acute T cell leukemia cells and folate-diketone in dose-dependent manner (effector: tumor cell ratio 5:1).

FIG. 9. Confocal imaging of CovCAR-T cells covalently modified by fluorescein-diketone. Nuclei were stained with Hoechst, catalytic antibody 38C2 scFv by the fluorescein-diketone, spacer domain of CAR was labeled by anti-IgG4 PE antibodies.

FIG. 10. Scheme of the chemical synthesis of one of the DUPA-diketone conjugates.

FIG. 11. In vivo activity of CovCAR-T cells and constant dosage of DUPA-3-diketone injections in a prostate cancer xenograft model. (A) NSG mice were implanted s.c. with 1 million PC3-PSMA cells. On day 3 after tumor inoculation 20×10⁶ of CovCAR-T cells with transduction efficacy of 60-75% were infused intravenously into animals from all experimental groups. Mice were then injected intravenously with either PBS or DUPA-3-diketone (500 nmol/kg) on day 3, 5, 7, 9, 11, 13, 15, 17. (B) Representative IVIS images of mice from placebo or DUPA-3-diketone treated groups. (C) Survival plots for animals from experimental and control groups. (D) Quantified tumor burden (as average radiance from luciferase activity per mouse) from (B) for 5-44 day period. (E) Individual animal tumor growth kinetics of mice from control and treated groups. Overall survival curves were plotted using the Kaplan-Meier method and compared using the log-rank (Mantel-Cox) test. Statistical significance: ** p<0.01. All data represent mean+SD.

FIG. 12. Effect of intraperitoneal administration of DUPA-3-diketone on the in vivo activity of CovCAR T cells in a prostate cancer xenograft model. (A) NSG mice were implanted s.c. with 1 million PC3-PSMA cells. On day 8 after tumor inoculation 20×10⁶ CovCAR-T cells or Mock transduced T cells were infused intravenously into animals from all experimental groups. Mice were then injected intraperitoneally with either PBS or DUPA-3-diketone (500 nmol/kg) every day for the next 26-days. (B) Representative IVIS images of the mice from all groups. (C) Tumor growth kinetics of mice from control and treated groups.

FIG. 13. Chemical structures for CovCAR-T with PSMA-specific diketone conjugates (DEL79/1, DEL79/2 and DEL1346/10-2-diketone) and synthesis of intermediates. (A) Chemical structures of the synthesized PSMA-specific diketone conjugates. All bispecific molecules have the same CovCAR recognition domains (diketone) with the same linker, and different PSMA recognition domain (DEL79/1-2-diketone, DEL79/2-2-diketone and DEL1346/10-2-diketone). (B) Synthesis of 1-(2-(bis((1-methyl-3-phenyl-1H-pyrazol-5-yl)methyl)amino)ethyl)-6-((1-methyl-3-phenyl-1H-pyrazol-5-yl)methyl)-4,5,6,7-tetrahydro-1H-pyrazolo[3,4-c]pyridine-3-carboxylic acid (DEL79/1-9) and 1-(2-(bis((1-methyl-3-phenyl-1H-pyrazol-5-yl)methyl)amino)ethyl)-6-(tert-butoxycarbonyl)-4,5,6,7-tetrahydro-1H-pyrazolo[3,4-c]pyridine-3-carboxylic acid (DEL79/2-2). (C) Synthesis of (2R)-2-(4-(3-((tert-butoxycarbonyl)amino) pyrrolidin-1-yl)-2-oxo-2H-chromene-7-carboxamido)-3-(3-methoxyphenyl) propanoic acid (DEL1346/10-7). (D) Synthesis of DEL79/1-2-diketone, DEL79/2-2-diketone and DEL1346/10-2-diketone.

FIG. 14. Chemical structures of the synthesized CovCAR-T with HER2-specific diketone conjugates (DEL675/1, DEL675/3, DEL675/5, DEL675/7, DEL675/9, DEL675/11-diketone) and synthesis of intermediates. (A) Chemical structures of the synthesized HER2-specific diketone conjugates. All bispecific molecules have the same CovCAR recognition domains (diketone) with the same linker (Linker 3 and Linker 4), and different PSMA recognition domain. (B) Synthesis of 6-(3-ethyl-4-(4-ethynylbenzoyl) piperazin-1-yl)-4-(4′-((4-ethynylbenzoyl)oxy)-[1,1′-biphenyl]-4-yl) nicotinic acid (DEL675-10), 6-(3-ethyl-4-(4-ethynylbenzoyl) piperazin-1-yl)-4-(4′-hydroxy-[1,1′-biphenyl]-4-yl) nicotinic acid (DEL675-20) and 6-(4-tert-butoxycarbonyl-3-ethyl-piperazin-1-yl)-4-[4-[4-(4-ethynylbenzoyl)oxyphenyl]phenyl]pyridine-3-carboxylic acid (DEL675-17). (C) Synthesis of 4-[4-(4-ethynylbenzoyl)-3-methyl-piperazin-1-yl]-3-[4-[4-(4-ethynylbenzoyl)oxyphenyl]phenyl]benzoic acid (DEL675-29), 4-(4-tert-butoxycarbonyl-3-methyl-piperazin-1-yl)-3-[4-[4-(4-ethynylbenzoyl)oxyphenyl]phenyl]benzoic acid (DEL675-35) and 4-[4-(4-ethynylbenzoyl)-3-methyl-piperazin-1-yl]-3-[4-(4-hydroxyphenyl)phenyl]benzoic acid (DEL675-38). (D) Synthesis of DEL675/1-3-diketone, DEL675/3-3-diketone, DEL675/5-3-diketone, DEL675/7-3-diketone, DEL675/9-3-diketone, DEL675/11-3-diketone, DEL675/1-4-diketone, DEL675/3-4-diketone, DEL675/5-4-diketone, DEL675/7-4-diketone, DEL675/9-4-diketone and DEL675/11-4-diketone.

### DETAILED DESCRIPTION

### I. Overview

The present invention is derived in part from studies undertaken by the inventors to the power of DNA-encoded libraries to select adapters for use in CAR-T therapy. As exemplified herein, the new paradigm developed by the inventors enabled construction of CAR-Ts that are effective against both breast and prostate cancer. In some embodiments, the adapters utilized by the inventors are organic compounds that contain two components. The first is a small molecule derived from the aforementioned DNA-encoded library that selectively binds to the tumor cell surface. The second is an appended substrate for a catalytic antibody that is a component of the CAR construct and allows the catalysis of covalent bond formation between the T cell and the tumor. Due to the covalent binding between the CAR-T cell and the adapter, it is less likely for them to dissociate at lower adaptor concentration. This could provide better stability of the complex as well as potential better efficacy when the CAR-T cells penetrate into tumor tissues. Additionally, in the case of catalytic antibody 38C2, the adapter binds to a site deeply buried in the antibody structure. It is unlikely for the antibody to bind to other undesired targets. As such, the employed organic compounds can bind specifically to tumor cell surface proteins, combined with a unique catalytic antibody whose safety has been validated in clinical trials as the CAR component and 1,3-diketone as the substrate functionality. DNA-encoded library technology allowed the inventors to overcome the scarcity of small molecules that bind specifically to surface proteins, and to successfully identify molecules specifically targeting the cancer cell surface. As such, the inventors were able to demonstrate that not only were the resultant CAR-Ts able to kill cancer cells both in vitro and in vivo, but also that their activity and toxicity could be controlled by exposure to the adapter. These exemplified experiments indicate this platform has the potential to be universally applicable for treating a wide range of cancers, while providing better control and an enhanced safety profile.

Catalytic antibodies were first introduced in the late 1980s. A few of them are particularly interesting in the context of CAR-T therapy because they are capable of catalyzing covalent bond formation with their substrates. One such antibody, the aldolase 38C2, has been used by pharmaceutical companies, tested in clinical trials, and proven to be safe in humans. Subsequently, this antibody has also been tested as a template for antibody drug conjugation, as well as other immunotherapies. The inventors used this antibody as part of a CAR assembly, the advantage being its prior use has already demonstrated its safety in the clinical setting. Under physiological conditions this catalytic antibody, which is an aldolase, forms a covalent bond with the small molecule 1,3-diketone, as well as other carbonyl-containing functionalities such as lactams. The inventors synthesized bifunctional adapter molecules that linked the selected cancer-targeting compounds obtained from DNA-encoded libraries (DELs) to the diketone. The result was that CAR-T function can be regulated by controlling the availability of the adapter molecule, thereby rendering CAR-T therapy subject to the usual pharmaco-kinetic controls characteristic of small molecule therapies. The smaller size of these adapter molecules offers rapid, reversible control of the CAR-T in vivo kinetics. Additionally, this construct allows T cells in the circulation to target the microenvironment of tumor cells more efficiently than ever before. We initially validated this concept with a few compounds known to bind selectively to the cancer cell surface. Subsequently, we isolated new targeting compounds from DELs.

DNA-encoded library technology dates to the early 1990's and was designed to select organic compounds targeting molecules of interest. Because of new sequencing technology, advances in the chemistry of DEL construction, and new information technologies for their de-convolution, DELs have recently taken a giant leap forward in terms of the capacity to screen heretofore unimaginable numbers of molecules in both a time- and resource-efficient manner. This has resulted in the current ability to successfully identify and select compounds with desired properties, no matter how rare they may be. The anti-tumor specificity of the CAR-T construct described here lies in the small molecule portion of the switchable adapter which selectively binds to the tumor cell surface. However, at present there are an extremely limited number of existing compounds known to bind cancer surface markers. We were able to use the power of DEL technology to overcome this problem and successfully identify compounds that selectively bind to the cell surface of the cancers we were studying. Heretofore, the conventional approach has been to select small molecules based on function. However, most cancer-specific cell surface proteins do not have easily quantifiable activities. For our purpose, we isolated compounds based solely on their ability to selectively bind to the target, because this is the only function needed to localize substrates for generation of covalent bonds. When coupled to diketone molecules, the synthesized small molecule adapters demonstrated great efficacy both in vitro and in vivo when incorporated into what we term covalent CAR-Ts (CovCAR-T).

Since the covalent bond-forming element of the CovCAR-T is universal, it can be engaged anew by simply using the power of DELs to change the small molecule portion of the adapter. This could be very useful clinically. For example, within the therapeutic course of a single patient this might be necessary if the original surface protein is down-regulated, if metastatic cells express a different surface protein or, less likely, if the patient develops a different type of cancer. Of course, the T cell component of a CovCAR-T is still subject to the usual histocompatibility restrictions. However, the bi-functional adapter controlling covalent bond formation and tumor cell specificity is truly universal.

In accordance with these studies, the present invention provides novel CAR-T compositions and their use in cancer therapies as described in detail below. Unless otherwise stated, the present invention can be performed using standard procedures, as described, for example in Methods in Enzymology, Volume 289: Solid-Phase Peptide Synthesis, J. N. Abelson, M. I. Simon, G. B. Fields (Editors), Academic Press; 1st edition (1997) (ISBN-13:978-0121821906); U.S. Pat. Nos. 4,965,343, and 5,849,954; Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2nd ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998).

The following sections provide additional guidance for practicing the compositions and methods of the present invention.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1st ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3rd ed., 2002); Dictionary of Chemistry, Hunt (Ed.), Routledge (1st ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4th ed., 2000). Further clarifications of some of these terms as they apply specifically to this invention are provided herein.

It is noted that, as used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

The term “antibody” typically refers to polypeptide chain(s) which exhibit a strong monovalent, bivalent or polyvalent binding to a given antigen, epitope or epitopes. Unless otherwise noted, antibodies or antibody fragments used in the invention also encompass certain immunoglobulin molecules or variant antibodies that do not possess specific antigen-binding activities, e.g., catalytic antibodies. immunoglobulin s can have sequences derived from any vertebrate species. They can be generated using any suitable technology, e.g., hybridoma technology, ribosome display, phage display, gene shuffling libraries, semi-synthetic or fully synthetic libraries or combinations thereof. Unless otherwise noted, the term “antibody” as used in the present invention includes intact antibodies, antibody fragments and other designer antibodies that are described below or well known in the art (see, e.g., Serafini, J Nucl. Med. 34:533-6, 1993).

An intact “antibody” typically comprises at least two heavy (H) chains (about 50-70 kD) and two light (L) chains (about 25 kD) inter-connected by disulfide bonds. The recognized immunoglobulin genes encoding antibody chains include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

Each heavy chain of an antibody is comprised of a heavy chain variable region (VH) and a heavy chain constant region. The heavy chain constant region of most IgG isotypes (subclasses) is comprised of three domains, CH1, CH2 and CH3, some IgG isotypes, like IgM or IgE comprise a fourth constant region domain, CH4 Each light chain is comprised of a light chain variable region (VL) and a light chain constant region. The light chain constant region is comprised of one domain, CL. The variable regions of the heavy and light chains contain a binding domain that interacts with an antigen. The constant regions of the antibodies may mediate the binding of the immunoglobulin to host tissues or factors, including various cells of the immune system and the first component (Clq) of the classical complement system.

The V_{H} and V_{L} regions of an antibody can be further subdivided into regions of hypervariability, also termed complementarity determining regions (CDRs), which are interspersed with the more conserved framework regions (FRs). Each V_{H} and V_{L} is composed of three CDRs and four FRs, arranged from amino-terminus to carboxyl-terminus in the following order: FR1, CDR1, FR2, CDR2, FR3, CDR3, FR4. The locations of CDR and FR regions and a numbering system have been defined by, e.g., Kabat et al., Sequences of Proteins of Immunological Interest, U.S. Department of Health and Human Services, U.S. Government Printing Office (1987 and 1991).

As used herein, an antibody fragment (or a fragment of an antibody) refers to any proteins or polypeptides that contain at least one antibody-derived V_{H}, V_{L}, or C_{H} immunoglobulin domain in the context of other non-immunoglobulin, or non-antibody derived components. Such molecules include, but are not limited to (i) Fc-fusion proteins of binding proteins, including receptors or receptor components with all or parts of the immunoglobulin CH domains, (ii) binding proteins, in which V_{H} and or V_{L} domains are coupled to alternative molecular scaffolds, or (iii) molecules, in which immunoglobulin V_{H}, and/or V_{L}, and/or CH domains are combined and/or assembled in a fashion not normally found in naturally occurring antibodies or antibody fragments. Unless otherwise noted, an antibody fragment contains the reactive amino acid residue of the antibody from which the fragment is derived.

“Humanized” forms of non-human (e.g., rodent, e.g., murine or rabbit) immunoglobulins are immunoglobulins which contain minimal sequences derived from non-human immunoglobulin. For the most part, humanized immunoglobulins are human immunoglobulins (recipient antibody) in which residues from a hypervariable region of the recipient are replaced by residues from a hypervariable region of a non-human species (donor antibody) such as mouse, rat, hamster, rabbit, chicken, bovine or non-human primate having the desired specificity, affinity, and capacity. In some instances, Fv framework region (FR) residues of the human immunoglobulin are also replaced by corresponding non-human residues. Furthermore, humanized antibodies can comprise residues which are not found in the recipient antibody or in the donor antibody. These modifications are made to further refine antibody performance. In general, the humanized immunoglobulin will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized immunoglobulin optionally also will comprise at least a portion of an immunoglobulin constant region (Fc), typically that of a human immunoglobulin. For further details, see Jones et al. (1986) Nature 321:522-525; Riechmann et al. (1988) Nature 332:323-329; and Presta (1992) Curr. Op. Struct. Biol. 2:593-596.

The term “human immunoglobulin”, as used herein, is intended to include immunoglobulins having variable and constant regions derived from human germline immunoglobulin sequences. The human immunoglobulins of the invention can include amino acid residues not encoded by human germline immunoglobulin sequences (e.g., mutations introduced by random or site-specific mutagenesis in vitro or by somatic mutation in vivo), for example in the CDRs and in particular CDR3. However, the term “human immunoglobulin”, as used herein, is not intended to include immunoglobulins in which CDR sequences derived from the germline of another mammalian species, such as a mouse, have been grafted onto human framework sequences.

The term “specific binding” or “specifically binds to” or is “specific for” refers to the binding of a binding moiety to a binding target, such as the binding of an immunoglobulin or small molecule agent to a target molecule or antigen, e.g., an epitope on a particular polypeptide, peptide, or other target (e.g. a glycoprotein target), and means binding that is measurably different from a non-specific interaction (e.g., a non-specific interaction can be binding to bovine serum albumin or casein). Specific binding can be measured, for example, by determining binding of a binding moiety (e.g., a small molecule agent), or an immunoglobulin, to a target molecule compared to binding to a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target.

The term “specific binding” or “specifically binds to” or is “specific for” a particular target molecule or an epitope on a particular target molecule can be exhibited, for example, by a molecule having a K_{d} for the target of at least about 200 nM, alternatively at least about 150 nM, alternatively at least about 100 nM, alternatively at least about 60 nM, alternatively at least about 50 nM, alternatively at least about 40 nM, alternatively at least about 30 nM, alternatively at least about 20 nM, alternatively at least about 10 nM, alternatively at least about 8 nM, alternatively at least about 6 nM, alternatively at least about 4 nM, alternatively at least about 2 nM, alternatively at least about 1 nM, or greater. In certain instances, the term “specific binding” refers to binding where a binding moiety binds to a particular target molecule or epitope on the target molecule without substantially binding to any other molecule or epitope.

The terms “conjugate,” “conjugated,” and “conjugation” refer to any and all forms of covalent or non-covalent linkage, and include, without limitation, direct genetic or chemical fusion, coupling through a linker or a cross-linking agent, and non-covalent association.

The term “fusion” is used herein to refer to the combination of amino acid sequences of different origin in one polypeptide chain by in-frame combination of their coding nucleotide sequences. The term “fusion” explicitly encompasses internal fusions, i.e., insertion of sequences of different origin within a polypeptide chain, in addition to fusion to one of its termini. The term “fusion” is used herein to refer to the combination of amino acid sequences of different origin.

The term “epitope” includes any molecular determinant capable of specific binding to an immunoglobulin. In certain aspects, epitope determinants include chemically active surface groupings of molecules such as amino acids, sugar side chains, phosphoryl, or sulfonyl, and, in certain aspects, can have specific three dimensional structural characteristics, and/or specific charge characteristics. An epitope is a region of an antigen that is bound by an immunoglobulin. A “binding region” is a region on a binding target bound by a binding molecule.

The term “target” or “binding target” is used in the broadest sense and specifically includes polypeptides, without limitation, nucleic acids, carbohydrates, lipids, cells, and other molecules with or without biological function as they exist in nature.

The term “antigen” refers to an entity or fragment thereof, which can bind to an immunoglobulin or trigger a cellular immune response. An immunogen refers to an antigen, which can elicit an immune response in an organism, particularly an animal, more particularly a mammal including a human. The term antigen includes regions known as antigenic determinants or epitopes, as defined above.

A nucleic acid is “operably linked” when it is placed in a functional relationship with another nucleic acid sequence. For example, DNA for a pre-sequence or secretory leader is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

“Percent (%) amino acid sequence identity” with respect to a peptide or polypeptide sequence, i.e., the h38C2 antibody polypeptide sequences or GCN4 derived peptides identified herein, is defined as the percentage of amino acid residues in a candidate sequence that are identical with the amino acid residues in the specific peptide or polypeptide sequence after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. Two sequences are “substantially identical” if they have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the well-known sequence comparison algorithms or by manual alignment and visual inspection.

“Treating” or “treatment” refers to both therapeutic treatment and prophylactic or preventative measures, wherein the object is to prevent or slow down (lessen) a targeted pathologic condition or disorder. Those in need of treatment include those already with the disorder, as well as those prone to have the disorder, or those in whom the disorder is to be prevented. For example, a subject or mammal is successfully “treated” for cancer, if, after receiving a therapeutic amount of a subject immunoconjugate according to the methods of the present invention, the subject shows observable and/or measurable reduction in or absence of one or more of the following: reduction in the number of cancer cells or absence of the cancer cells; reduction in the tumor size; inhibition (i.e., slowing to some extent and preferably stopping) of cancer cell infiltration into peripheral organs, including the spread of cancer into soft tissue and bone; inhibition (i.e., slowing to some extent and preferably stopping) of tumor metastasis; inhibition, to some extent, of tumor growth; and/or relief to some extent of one or more of the symptoms associated with the specific cancer; reduced morbidity and/or mortality, and improvement in quality of life issues.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” refer to a variant which has conservative amino acid substitutions, amino acid residues replaced with other amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The term “contacting” has its normal meaning and refers to combining two or more agents (e.g., polypeptides or phage), combining agents and cells, or combining two populations of different cells. Contacting can occur in vitro, e.g., mixing an antibody and a cell or mixing a population of antibodies with a population of cells in a test tube or growth medium. Contacting can also occur in a cell or in situ, e.g., contacting two polypeptides in a cell by co-expression in the cell of recombinant polynucleotides encoding the two polypeptides, or in a cell lysate. Contacting can also occur in vivo inside a subject, e.g., by administering an agent to a subject for delivery the agent to a target cell.

The term “subject” refers to human and non-human animals (especially non-human mammals). The term “subject” is used herein, for example, in connection with therapeutic and diagnostic methods, to refer to human or non-human animals. Specific examples of non-human subjects include, e.g., cows, horses, sheep, pigs, cats, dogs, mice, rats, rabbits, guinea pigs, monkeys.

Artificial T cell receptors (also known as chimeric T cell receptors, chimeric immunoreceptors, chimeric antigen receptors (CARs) or T-bodies) are engineered receptors, which graft an arbitrary specificity onto an immune effector cell. Typically, these receptors are used to graft the specificity of a monoclonal antibody onto a T cell; with transfer of their coding sequence facilitated by retroviral or lentiviral vectors or by transposons. CAR-engineered T cells (also abbreviated CAR-T cells) are genetically engineered T cells armed with chimeric receptors whose extracellular recognition unit is comprised of an antibody-derived recognition domain and whose intracellular region is derived from one or more lymphocyte stimulating moieties. The structure of the prototypic CAR is modular, designed to accommodate various functional domains and thereby to enable choice of specificity and controlled activation of T cells. The preferred antibody-derived recognition unit is a single chain variable fragment (scFv) that combines the specificity and binding residues of both the heavy and light chain variable regions of a monoclonal antibody. The most common lymphocyte activation moieties include a T-cell costimulatory (e.g. CD28 and/or 4-1BB) domain in tandem with a T-cell triggering (e.g. CD3zeta) moiety. By arming effector lymphocytes (such as T cells and natural killer cells) with such chimeric receptors, the engineered cell is re-directed with a pre-defined specificity to any desired target antigen, in a non-HLA restricted manner. CAR constructs are introduced ex vivo into T cells from peripheral lymphocytes of a given patient using retroviral or lentiviral vectors or transposons. Following infusion of the resulting CAR-engineered T cells back into the patient, they traffic, reach their target site, and upon interaction with their target cell or tissue, they undergo activation and perform their predefined effector function. Therapeutic targets for the CAR approach include cancer and HIV-infected cells, or autoimmune effector cells.

A “vector” is a replicon, such as plasmid, phage or cosmid, to which another polynucleotide segment may be attached so as to bring about the replication of the attached segment. Vectors capable of directing the expression of genes encoding for one or more polypeptides are referred to as “expression vectors”.

### III. Covalent CAR-Ts Containing Catalytic Antibodies

The invention provides covalent CAR-T (CovCAR-T) compositions that contain (1) a CAR-T cell with an catalytic antibody in the extracellular domain of its CAR and (2) an adapter compound containing a targeting moiety that is fused to a substrate moiety of the catalytic antibody. When the CAR-T cell is contacted with the adapter compound, the antibody catalyzes formation of a covalent bond between the substrate moiety and a reactive residue in the binding site of the antibody. By fusing the substrate to different targeting moieties, the adapter molecule functions as a switch to direct the inert CAR-T cell to different cells that are recognized by the different targeting moieties.

Any known catalytic antibody or antibody fragment thereof can be used in the construction of the CAR-T cell in the invention. Catalytic antibodies, also called “abzyme” or “catmab,” are monoclonal antibodies with catalytic activities, e.g., the well-known catalytic antibody 38C2. Catalytic antibodies are usually artificial constructs, but are also found in healthy subjects and some patients, e.g., certain autoantibodies. Various catalytic antibodies may be employed in the practice of the invention. Examples of include aldolase antibodies, beta lactamase antibodies, esterase antibodies, amidase antibodies, and the like. These antibodies usually have reactive amino acid amino acid side chain in the combining site which is involved in their catalytic functions. For example, they can have a reactive lysine residue, reactive cysteine, serine, or tyrosine residue.

In some embodiments, the catalytic antibody to be employed in the invention is an aldolase antibody. Aldolase catalytic antibodies are antibodies which exhibit aldolase activities (e.g., catalyzes of the aldol reaction). The aldol reaction is an important carbon-carbon bond formation reaction in organic chemistry. In its usual form, it involves the nucleophilic addition of a ketone enolate to an aldehyde to form a β-hydroxy ketone, or “aldol” (aldehyde+alcohol). Examples of aldolase catalytic antibodies include 38C2 and 33F12 as reported, e.g., in Wagner et al., Science 270:1797, 1995. These antibodies, which were obtained via reactive immunization, exhibit high efficiency and enantioselectivity over a broad range of ketone and aldehyde substrates. They utilize a reactive lysine residue and catalyze the aldol as well as the retrograde aldol reactions under neutral pH using the enamine mechanism of natural aldolases. See, e.g., Barbas et al., Science 278:2085-2092, 1997; Zhong et al., Angew Chem Int Ed Engl. 38:3738-3741, 1999; Karlstrom et al., Proc. Natl. Acad. Sci. USA. 97:3878-3883, 2000; and Hoffmann et al., J. Am. Chem. Soc. 120:2768, 1998. In addition to their versatility and efficacy in synthetic organic chemistry (e.g., Hoffmann et al., J. Am. Chem. Soc. 120:2768-2779, 1998; and Sinha et al., Proc. Natl. Acad. Sci. USA 95:14603-14608, 1998), aldolase antibodies have been used to activate camptothecin, doxorubicin, and etoposide prodrugs in vitro and in vivo as an anti-cancer strategy (Shabat et al., Proc. Natl. Acad. Sci. USA 96:6925-6930, 1999; and Shabat et al., Proc. Natl. Acad. Sci. USA 98:7528-7533, 2001).

In some preferred embodiments, the antibody to be used in the CAR-T cell of the invention is derived from catalytic antibody 38C2, its variants or an antibody fragment thereof. The 38C2 catalytic antibody and its humanized variant are well known in the art and extensively characterized in the art, e.g., Wagner et al., Science 270, 1797-1800, 1995; Barbas, et al., Science 278, 2085-2092, 1997; Rader et al., J. Mol. Biol. 332, 889-899, 2003; Nanna et al., Nat. Commun. 8, 1112, 2017; Hwang et al., Cell Chem. Biol. 26, 1229-1239, 2019; and U.S. Pat. No. 8,252,902. The heavy chain variable region of the 38C2 antibody includes a single, uniquely reactive lysine residue (Lys99) that can react with a linker, thereby providing an attachment point for conjugation with a drug moiety. As such, immunoglobulin molecules that include a variable domain of the 38C2 antibody contain two such attachment points (one on each heavy chain) that can be used for conjugation with a substrate moiety or drug moiety. Once a reactive lysine residue has been conjugated to a linker, the 38C2 antibody no longer exhibits catalytic activity. There have been a number of examples of antibody-conjugated drug compounds generated by the use of the reactive lysine residue (Lys99) in the active site of catalytic antibody 38C2 for site-specific bioconjugation. See, e.g., Rader, Proc. Natl. Acad. Sci. U.S.A 100, 5396-5400, 2003; Rader, Trends Biotechnol 32, 186-197, 2014; Nanna et al., Nat. Commun. 8, 1112, 2017; Qi et al., Front. Immunol. 10, 1994, 2019; Hwang et al., Bioconjug. Chem. 30, 2889-2896, 2019; and U.S. Pat. No. 8,252,902.

Antibodies suitable for use herein may be obtained by conventional immunization, reactive immunization in vivo, or by reactive selection in vitro, such as with phage display. Antibodies may be produced in humans or in other animal species. Antibodies from one species of animal may be modified to reflect another species of animal. For example, human chimeric antibodies are those in which at least one region of the antibody is from a human immunoglobulin. A human chimeric antibody is typically understood to have variable regions from a non-human animal, e.g. a rodent or a lagomorph, with the constant regions from a human. In contrast, a humanized antibody uses CDRs from the non-human antibody with most or all of the variable framework regions from and all the constant regions from a human immunoglobulin. Chimeric and humanized antibodies may be prepared by methods well known in the art including CDR grafting approaches (see, e.g., U.S. Pat. Nos. 5,843,708; 6,180,370; 5,693,762; 5,585,089; 5,530,101), chain shuffling strategies (see e.g., U.S. Pat. No. 5,565,332; Rader et al., Proc. Natl. Acad. Sci. USA (1998) 95:8910-8915), molecular modeling strategies (U.S. Pat. No. 5,639,641), and the like.

While a full length catalytic antibody (e.g., 38C2) or antibody fragment thereof may be employed in the practice of the invention, a single chain configuration of the antibody is more preferred in construction the CAR of the CAR-T cell. In some embodiments, a single chain variable fragment (scFv) of the catalytic antibody can be employed in the construction of the CAR-T cell. In these embodiments, while the scFv is located at the N-terminus of the CAR, it can either have the light chain sequence or the heavy chain sequence located at the N-terminus, as exemplified herein. In some preferred embodiments, the scFv in the extracellular domain of the CAR has the V_{L} sequence at the N-terminus, which is linked at is C-terminus to the V_{H} sequence. As exemplified herein, the light chain and heavy chain variable regions of the antibody (e.g., 38C2) can be readily linked via a suitable linker or spacer. Various linker moieties can be used to generate the scFv fragments. In some embodiments, a Gly-Ser linker can be used.

In some preferred embodiments, the employed scFv is derived from antibody 38C2. In some preferred embodiments, the scFv is generated from a humanized 38C2 antibody (h38C2) or antibody fragment thereof. In some preferred embodiments, the catalytic antibody for constructing the CovCAR-T of the invention is h38C2 antibody or a binding fragment thereof that contains heavy chain and light chain variable region sequences shown below or conservatively modified variants thereof. In some of these embodiments, a Ser-Gly linker can be used to connect the light chain sequence to the heavy chain sequence. An example of such linker is GGGGSGGGGSGGGGS (SEQ ID NO:3) as exemplified herein.

V_{κ} of h38C2 (SEQ ID NO: 1):
DVVMTQTPLSLPVRLGDQASISCRSSQSLLHTYGSPYLNWYLQKPGQSP
KLLIYKVSNRFSGVPDRFSGSGSGTDFTLRISRVEAEDLGVYFCSQGTH
LPYTFGGGTKLEIKR
V_{H} of h38C2 (SEQ ID NO: 2):
EVKLVESGGGLVQPGGTMKLSCEISGLTFRNYWMSWVRQSPEKGLEWVA
EIRLRSDNYATHYAESVKGKFTISRDDSKSRLYLQMNSLRTEDTGIYYC
KTYFYSFSYWGQGTLVTVS

A specific CovCAR molecule bearing a h38C2 scFv in the extracellular domain is shown in SEQ ID NO:14. In this sequence, an IL-2 signal peptide sequence (MYRMQLLSCIALSLALVINS; SEQ ID NO:13) is present at the N-terminus, which is followed by the h38C2 scFv sequence.

h38C2 scFv CovCAR molecule (SEQ ID NO: 14):
MYRMQLLSCIALSLALVTNSDVVMTQTPLSLPVRLGDQASISCRSSQSL
LHTYGSPYLNWYLQKPGQSPKLLIYKVSNRFSGVPDRFSGSGSGTDFTL
RISRVEAEDLGVYFCSQGTHLPYTFGGGTKLEIKRGGGGSGGGGSGGGG
SEVKLVESGGGLVQPGGTMKLSCEISGLTFRNYWMSWVRQSPEKGLEWV
AEIRLRSDNYATHYAESVKGKFTISRDDSKSRLYLQMNSLRTEDTGIYY
CKTYFYSFSYWGQGTLVTVSESKYGPPCPSCPAPEFEGGPSVFLFPPKP
KDTLMISRTPEVTCVVVDVSQEDPEVQFNWYVDGVEVHNAKTKPREEQF
QSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSL
SLGKGSTSGSGKPGSGEGSTKGFWVLVVVGGVLACYSLLVTVAFIIFWV
RSKRSRLLHSDYMNMTPRRPGPTRKHYQPYAPPRDFAAYRSRDQRLPPD
AHKPPGGGSFRTPIQEEQADAHSTLAKIRVKFSRSADAPAYQQGQNQLY
NELNLGRREEYDVLDKRRGRDPEMGGKPRRKNPQEGLYNELQKDKMAEA
YSEIGMKGERRRGKGHDGLYQGLSTATKDTYDALHMQALPPR

As exemplified, the employed scFv fragment can be connected to the other domains of the CAR of the CAR-T cell via standard recombinant techniques well known in the art. In some embodiments, the scFv is conjugated to the transmembrane domain of the CAR via a spacer or hinge domain to provide flexibility and to facilitate interaction of the scFv with its substrate. In some embodiments, the space domain comprises CH2-CH3 IgG4 as exemplified herein. Other suitable spacer or hinge domains for connecting the scFv to the transmembrane domain are also exemplified below.

CH2-CH3 IgG4 spacer domain (SEQ ID NO: 4):
APEFEGGPSVFLFPPKPKDTLMISRTPEVTCVVVDVSQEDPEVQFNWYV
DGVEVHNAKTKPREEQFQSTYRVVSVLTVLHQDWLNGKEYKCKVSNKGL
PSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCS
VMHEALHNHYTQKSLSLSLGK
IgG4 hinge (SEQ ID NO: 5):
ESKYGPPCPSCP
CD8a hinge domain (SEQ ID NO: 6):
TTTPAPRPPTPAPTIASQPLSLRPEACRPAAGGAVHTRGLDFAC
IgG4 CH2 domain (SEQ ID NO: 7):
APEFEGGPSVFLFPPKPKDTLMISRTPEVTCVVVDVSQEDPEVQFNWYV
DGVEVHNAKTKPREEQFQSTYRVVSVLTVLHQDWLNGKEYKCKVSNKGL
PSSIEKTISKAK
IgG4 CH3 domain (SEQ ID NO: 8):
GQPREPQVYTLPPSQEEMTKNQVSLTCLVKGFYPSDIAVEWESNGQPEN
NYKTTPPVLDSDGSFFLYSRLTVDKSRWQEGNVFSCSVMHEALHNHYTQ
KSLSLSLGK

In some embodiments, there can be a second linker between the hinge or spacer and the transmembrane to provide additional flexibility. An example of such a second linker, GSTSGSGKPGSGEGSTKG (SEQ ID NO:9), is present in the CovCAR molecule exemplified herein.

Transmembrane and intracellular domain sequences that are suitable for constructing the CovCAR T cells of the invention are well known in the art. These include the CD28 transmembrane and intracellular domain, OX-40 intracellular domain, and the CD3z intracellular domain that are present in the h38C2 scFv CovCAR molecule exemplified herein. Construction of the CovCAR molecule of the invention with the various component motifs or domains can be readily performed in accordance with standard recombinant techniques well known in the art or specific guidance described herein.

CD28 transmembrane and intracellular domain (SEQ
ID NO: 10):
FWVLVVVGGVLACYSLLVTVAFIIFWVRSKRSRLLHSDYMNMTPRRPGP
TRKHYQPYAPPRDFAAYRS
OX-40 intracellular domain (SEQ ID NO: 11):
RDQRLPPDAHKPPGGGSFRTPIQEEQADAHSTLAKI
CD3z intracellular domain (SEQ ID NO: 12):
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

### IV. Adapter Molecules Containing a Substrate Moiety for Forming CovCAR-Ts

To target the CAR-T cells to different cells of interest, the CovCAR-T compositions of the invention employ an adapter compound (“adapter molecule” or “switch compound”). In the adapter molecule, a targeting moiety is fused to a substrate moiety of the catalytic antibody in the extracellular domain of the CAR of the CAR-T cell. Catalyzed by the catalytic antibody, the substrate moiety is able to form a covalent bond with the reactive residue of the antibody, thereby providing targeting specificity to the CAR-T cell.

Any know substrate moieties of a catalytic antibody can be used in constructing the adapter molecules of the invention. When antibody 38C2 is employed in the CAR-T construction, formation of a covalent bond between the targeting moiety-linked substrate to the reactive amino acid residue (e.g., Lys99) of the 38C2 antibody or 38C2 variant can be achieved via any known substrate moiety of a reactive Lys residue. These include, e.g., a 1,3-diketone, β-lactam based linker moiety and a heteroaryl methylsulfonyl compound. Various heteroaryl methylsulfonyl compounds can be employed in the practice of the invention. These include many methylsulfonyl 5-member monocyclic compounds, such as phenyltetrazoles or phenyloxadiazoles, which are well known in the art. See, e.g., Toda et al., Angew Chem Int Ed Engl, 52:12592-6, 2013; and Patterson et al., Bioconjug Chem, 25:1402-7, 2014. In some embodiments, the heteroaryl methylsulfonyl compound for functionalizing antibody 38C2 or derivatizing an agent moiety (e.g., a drug compound) is methylsulfone phenyloxadiazole (MS-PODA). See, e.g., Hwang et al., Bioconjug. Chem. 30, 2889-2896, 2019.

In some other embodiments, the extracellular domain of the CAR of the CAR-T cell can have a catalytic antibody containing a reactive amino acid residue that is cysteine, serine, arginine or tyrosine residue. Reactive cysteines may be found in thioesterase catalytic antibodies as described in, e.g., Janda et al., Proc. Natl. Acad. Sci. USA 91:2532-2536, 1994; and Wirsching et al., Science 270:1775-82, 1995. These antibodies may form a covalent linkage with maleimide-containing components or other thiol-reactive groups such as iodoacetamides, aryl halides, disulfhydryls and the like. The adapter molecule in these embodiments can contain a substrate moiety having one of these functional groups.

Conjugation of the substrate moiety to the targeting moiety in the adapter molecule can be achieved via routinely practiced chemical techniques. Using antibody 38C2 as an example, the targeting moiety can be fused to one of the known substrate moieties (diketone, β-lactam or heteroaryl methylsulfonyl) that is able to form a covalent bond with the reactive lysine in the antibody. Any compounds that contain or are functionalized by one of the substrate moiety can be used in constructing the adapter molecule. For example, when diketone is used as the substrate moiety, a diketone derivatized RGD peptidomimetic may be used. See, e.g., Rader et al., PNAS Apr. 29, 2003 100 (9) 5396-5400; and Li et al., J. Med. Chem. 2004, 47, 5630-5640. Similarly, adapter molecules containing a target moiety that fused to one of the other known substrate moieties of antibody 38C2 (such as lactam or) can also be synthesized in accordance with experimental protocols that have been reported in the literature. See, e.g., Gavrilyuk et al., Bioorg Med Chem Lett. 2009; 19 (5): 1421-1424; Nanna et al., Nat. Comm. 2017; 8:1112; Hwang et al., Bioconjug Chem. 2019; 30 (11): 2889-2896; and PCT publication WO2021/080846.

In various embodiments, other than the substrate moiety, the compound that is fused to the targeting moiety in the adapter molecule can additionally contain a linker moiety or side chain for connecting the substrate moiety to the targeting moiety. As exemplified herein with PMSA-targeting DUPA-diketone adapter molecules, a suitable linker will provide an appropriate space between the CAR-T cell and the targeting moiety for optimal cytotoxic activities once the CovCAR-T engages the target cell.

### V. Targeting Moieties for Target-Specific Delivery of CAR-T

The adapter molecules for covalently bonding to the CAR-T cell of the invention contains a substrate moiety of the catalytic antibody in the extracellular domain of the CAR, which is fused to a targeting moiety that specifically recognizes a target molecule (e.g., a tumor antigen) present on the surface of a target cell of interest (e.g., a tumor cell). “Targeting agent” or “targeting moiety” as used herein refers to a moiety that recognizes, binds or adheres to a target molecule located in a cell, tissue (e.g. extracellular matrix), fluid, organism, or subset thereof. Preferably, the target molecule is a molecule located on the surface of the target cell, esp. a tumor cell surface receptor. In some embodiments, the adapter molecule can contain more than one targeting moiety or targeting agent. For example, one of the targeting moieties can bind specifically to one target molecule on the target cell, and a second targeting moiety can bind specifically to another target molecule present on the same or different target cell.

A targeting moiety and its cognate target molecule represent a binding pair of molecules, which interact with each other through any of a variety of molecular forces including, for example, ionic, covalent, hydrophobic, van der Waals, and hydrogen bonding, so that the pair have the property of binding specifically to each other. Specific binding means that the binding pair exhibit binding with each other under conditions where they do not bind to another molecule. Examples of binding pairs are biotin-avidin, hormone-receptor, receptor-ligand, enzyme-substrate, lgG-protein A, antigen-antibody, and the like. The targeting moiety and its cognate target molecule exhibit a significant association for each other. This association may be evaluated by determining an equilibrium association constant (or binding constant) according to methods well known in the art. Affinity is calculated as K_{d}=k_{off}/kₒₙ (k_{off} is the dissociation rate constant, kₒₙ is the association rate constant and K_{d} is the equilibrium constant.

In the practice of the invention, targeting moieties include, but are not limited to, small molecule organic compounds of 5,000 daltons or less such as drugs, peptides, peptidomimetics, proteoglycans, lipids glycolipids, phospholipids, lipopolysaccharide, nucleic acids, carbohydrates, and the like. In some preferred embodiments, the targeting moiety is a small molecule as compared with a native immunoglobulin. The targeting moiety or targeting agent, including any linking moiety necessary for covalently linking the targeting moiety to an amino acid residue of the antibody, preferably is at least about 300 daltons in size, and preferably may be at least about 400, 500, 600, 700, 800, 900, 1,000, 1,100, 1,200, 1,300, 1,400, 1,500, 1,600, 1,700, 1,800, 1,900, 2,000, 2,500, 3,000, 3,500, 4,000, 4,500 or even 5,000 daltons in size, with even larger sizes possible.

In some embodiments, the small molecule targeting moieties are compounds known to bind to a target molecule of a target cell (e.g., a tumor cell). Specific examples include, but are not limited to, folate and 2-[3-(1,3-dicarboxypropyl) ureido]pentanedioic acid (DUPA) as exemplified herein for targeting folate receptor-expressing cancer cells and prostate cancer cells, respectively. Other examples of suitable targeting agents include RGD and other short peptides and peptidomimetics that bind to av integrins, peptidomimetic LLP2A binding to integrin a4β1, sialic acid derivatives targeting Siglec family members, and chemokine mimetics targeting chemokine receptors. In still some other embodiments, small molecule agents for generating adapter switches of the invention can also be compounds without known target binding activities. For example, compounds in any known small molecule compound libraries can be used for producing the adapter molecules in the practice of the invention. In some embodiments, the targeting agents can be obtained from a commercially available drug-like small molecule compound library, e.g., Chembridge DIVERSet, Maybridge HitFinder and GlycoNet collection from SPARC BioCentre (Toronto, Canada), and DNA-encoded library from WuXi AppTec (Shanghai, China).

In some embodiments, small molecule targeting moieties suitable for the invention can be compounds that are known to bind to a target molecule on the surface of a target cell (e.g., a tumor cell). Examples of such targeting agents include various known drugs that target tumor surface receptors such as EGFR, VEGFR1-3, PDGFR, HGFR. Specific examples include small molecule drugs Imanitib, Gefitinib, Erlotinib, Sunitinib, Lapatinib, Nilotinib, Temsirolimus, Sorafenib, Everolimus, Pazopanib, Crizotinib, Ruxolitinib, Axitinib, Bosutinib, Cabozantinib, Ponatinib, Regorafenib, Ibrutinib, Trametinib, and Perifosine. Other suitable known small molecule drugs include small molecules that target matrix metalloproteinases and cell surface heat shock proteins.

VI. Selecting Novel Targeting Moieties from DNA-Encoded Libraries

In addition to know small molecule compounds that specifically target tumor cells, additional targeting moiety compounds to be used in the invention can be obtained by screening DNA-encoded compound libraries. DNA-encoded libraries (DELs) are collections of small molecules covalently attached to amplifiable DNA tags carrying unique information about the structure of each library member. A combinatorial approach is used to construct the libraries with iterative DNA encoding steps, facilitating tracking of the synthetic history of the attached compounds by DNA sequencing. Various screening protocols have been developed which allow protein target binders to be selected out of pools containing up to billions of different small molecules. The versatile methodology has allowed identification of numerous biologically active compounds and is now increasingly being adopted as a tool for lead discovery campaigns and identification of chemical probes. A great focus in recent years has been on developing DNA compatible chemistries that expand the structural diversity of the small molecule library members in DELs.

In the practice of the invention, screening of DELs for novel targeting moieties can be readily performed in accordance with methods well known in the art. The principle of tagging combinatorial libraries with DNA was first described by Brenner and Lerner, Proc Natl. Acad. Sci. USA, 89 (1992), pp. 5381-5383. In the report, libraries of peptides were being synthesized on a solid support from beads containing two orthogonally protected linkers. Alternating oligonucleotide and peptide synthesis was carried out and thus the DNA coding was done chemically. Exploiting the split- and-pool approach, DELs of up to 10⁶ members were synthesized on solid support. A few years later, an enzymatic ligation approach for the introduction of DNA codes in a combinatorial setting was reported (Kinoshita and Nishigaki, Nucleic Acids Symp Ser, 34 (1995), pp. 201-202). Consequently, the compound synthesis was no longer required to be compatible with the subsequent oligonucleotide synthesis and the DEL synthesis was not restricted to solid support. Hence, the encoding could be created by iterative ligations, and the process of alternating chemical modification and enzymatic ligation in solution has been widely used for the synthesis of DELs. See, e.g., Clark et al., Nat Chem Biol, 5 (2009), pp. 647-654; Deng et al., J Med Chem, 55 (2012), pp. 7061-7079; Ding et al., ACS Comb Sci, 20 (2018), pp. 251-255; Ding et al., ACS Comb Sci, 18 (2016), pp. 625-629; Stress et al., Angew Chem. Int Ed, 58 (2019), pp. 9570-9574; Deng et al., ACS Med Chem Lett, 6 (2015), pp. 919-924; and Fan et al., ChemBioChem, 18 (2017), pp. 843-847. Additional methods for the encoding of DELs in which unique DNA tags are formed and covalently connected to a small molecule are described in, e.g., Shi et al., Bioorg Med Chem Lett, 27 (2017), pp. 361-369; and Favalli et al., FEBS Lett, 592 (2018), pp. 2168-2180.

In some embodiments, synthesis of DELs can be performed with DNA-recorded library synthesis as described in, e.g., Clark et al., Nat Chem Biol, 5 (2009), pp. 647-654; and Mannocci et al., Proc. Natl. Acad. Sci. USA, 105 (2008), pp. 17670-17675. In these methods, the libraries are prepared by split- and-pool synthesis where a DNA strand specific for a given BB is ligated after a chemical reaction has taken place. The starting point for library assembly is typically an amino-modified double stranded DNA (dsDNA) construct, and the codon ligation is carried out using ds codon/anti-codon duplexes with overhangs, which are ligated enzymatically by either polymerases or ligases. In some embodiments, splint ligation procedures can be employed for DNA-recorded libraries with single stranded DNA (ssDNA), where the final dsDNA construct is formed via a Klenow polymerization step. See, e.g., Li et al., Nat Chem, 10 (2018), pp. 441-448; and Leimbacher et al., Chem Eur J, 18 (2012), pp. 7729-7737.

In some other embodiments, DNA-directed libraries can be used in the practice of the invention. In contrast to DNA-recorded libraries, DNA-directed chemistry is a concept where the encoding DNA strand not only serves to track the synthetic history of the attached small molecule, but it also has an additional role of directing the chemistry of each step. Thus, the power of DNA-hybridization is used to achieve a high proximity effect of the building blocks and allow the reaction to take place in low DNA concentrations, much like an intramolecular reaction. See, e.g., Gartner et al., Science, 305 (2004), pp. 1601-1605; Tse et al., J Am Chem Soc, 130 (2008), pp. 15611-15626; Usanov et al., Nat Chem, 10 (2018), pp. 704-714; and Kleiner et al., J Am Chem Soc, 132 (2010), pp. 11779-11791. In some other embodiments, an encoded self-assembling combinatorial library can be employed in the invention. See, e.g., Melkko et al., Angew Chem Int Ed, 119 (2007), pp. 4755-4758; Melkko et al., Chem Biol, 13 (2006), pp. 225-231; Wichert et al., Nat Chem, 7 (2015), pp. 241-249; and Bigatti et al., ChemMedChem, 12 (2017), pp. 1748-1752. In still some other embodiments, PNA-encoded libraries can be used. Construction and screening of PNA-encoded compound libraries can be performed as described in, e.g., Zambaldo et al., Curr Opin Chem Biol, 26 (2015), pp. 8-15.

There are various advantages associated with DELs for identifying novel targeting compounds for the invention. In traditional HTS, each individual compound in the library is discretely screened under predefined assay conditions. DEL hits, on the contrary, are identified from mixtures of compounds and selected out of a pool by an affinity selection. Furthermore, DEL screenings are more amenable to testing several binding conditions in parallel as each experiment takes place in only a single tube. After isolating the DNA from the selection, this is PCR amplified and upon high-throughput DNA sequencing, the target binders are identified from the counted sequences. The structure of the hits can then be deconvoluted through translation of the corresponding DNA ‘barcode’ and compounds are subsequently synthesized off-DNA to confirm their activity in biological assays. The technique enables the screening of compound collections containing more than a billion distinct members, and successful screens are routinely carried out with only atto-to femtomoles of each tagged library member. See, e.g., Satz, ACS Med Chem Lett, 9 (2018), pp. 408-410. Indeed, very small quantities of both library and target are required which, in comparison to the widely used HTS, offers a more rapid and simple screening approach with significantly reduced cost and time investments. Utilizing the DNA-encoded libraries, novel targeting moieties suitable for the CovCAR-T compositions of the invention can be readily identified that specifically target the surface of various cancer cells. This is exemplified herein with targeting moieties for PSMA- and HER2-expressing cancers.

### VI. Therapeutic Applications and Pharmaceutical Compositions

The invention provides methods for using the CovCAR-T compositions described herein for targeting CAR-T cells to a cellular site (e.g., a target molecule on the surface of a target tumor cell). As noted above, the CAR-T cell used in these therapeutic applications has a catalytic antibody in the extracellular binding domain of its chimeric antigen receptor. When the CAR-T cells is co-administered with an adapter molecule, the antibody can catalyze formation of a covalent bond between its reactive amino acid and a targeting moiety in the adapter molecule.

The generic and inert CAR-T cells which can covalently bind to and be activated by the co-administered adapter molecule can be prepared in accordance with methods well known in the art or specific protocols exemplified in, e.g., WO2018/075807, WO2015057834, WO2015057852, and Marcu-Malina et al., Expert Opinion on Biological Therapy, Vol. 9, No. 5. In general, the catalytic antibody, preferably in a single chain format, is linked to a synthetic molecule containing one or more of the following domains: a spacer or hinge region (e.g., a CD28 sequence or a IgG4 hinge-Fc sequence), a transmembrane region (e.g., a transmembrane canonical domain), and an intracellular T-cell receptor (TCR) signaling domain, thereby forming a chimeric antigen receptor (CAR) or T-body. Intracellular TCR signaling domains that can be included in a CAR (or T-body) include, but are not limited to, CD3ζ, FcR-γ, and Syk-PT signaling domains as well as the CD28, 4-1BB, and CD134 co-signaling domains.

Recombinant technology can be used to introduce CAR-encoding genetic material into any suitable T-cells, e.g., human T cells such as central memory T-cells. As exemplification, the CAR-T cells to be used in the methods of the invention can be generated by transduction of human T cells with a lentiviral vector expressing the engineered CAR. The lentiviral vector can contain an expression cassette that fuses the sequences of a scFv catalytic antibody (e.g., a 38C2 scFv) to a human IgG4 hinge, followed by the transmembrane segment of CD28 and the cytoplasmic signaling domains of 4-1BB and CD3ζ. Downstream of the CAR and linked by a T2A ribosomal skip element, the vector can further encode a truncated epidermal growth factor receptor (EGFRt) sequence. The binding domain of the CAR (e.g., scFv 38C2) can have a GS linker to connect the variable region of the light chain (VL) and the variable region of the heavy chain (VH). For example, a Gly-Ser linker can be used to link the two variable region sequences of the antibody. Upon transduction of the CAR-expressing lentiviral vector into T cells, CAR-T cells can be expanded from PBMCs ex vivo (e.g., in the presence of cytokines).

When the adapter molecule and the otherwise inert CAR-T cells are co-administered to a subject in need of treatment, the CAR-T cells become covalent bound by the adapter molecule, and are delivered to the cellular site recognized by the targeting moiety in the adapter. Administration of the adapter and the CAR-T cells can be simultaneous or sequential. The administration can be performed in accordance with standard protocols of immunotherapy. In some preferred embodiments, adapter and the CAR-T cells are administered to the subject by infusion.

The invention further provides pharmaceutical compositions that contain an adapter molecule and/or a CAR-T cell described herein and a pharmaceutically acceptable carrier. Pharmaceutical compositions can be prepared from any of the adapter molecules and inert CAR-T cells described herein. The pharmaceutically acceptable carrier can be any suitable pharmaceutically acceptable carrier. It can be one or more compatible solid or liquid fillers, diluents, other excipients, or encapsulating substances which are suitable for administration into a human or veterinary patient (e.g., a physiologically acceptable carrier or a pharmacologically acceptable carrier). The term “carrier” denotes an organic or inorganic ingredient, natural or synthetic, with which the active ingredient is combined to facilitate the use of the active ingredient, e.g., the administration of the active ingredient to a subject. The pharmaceutically acceptable carrier can be co-mingled with one or more of the active components, e.g., an adapter molecule, and with each other, when more than one pharmaceutically acceptable carrier is present in the composition, in a manner so as not to substantially impair the desired pharmaceutical efficacy. Pharmaceutically acceptable materials typically are capable of administration to a subject, e.g., a patient, without the production of significant undesirable physiological effects such as nausea, dizziness, rash, or gastric upset. It is, for example, desirable for a composition comprising a pharmaceutically acceptable carrier not to be immunogenic when administered to a human patient for therapeutic purposes.

Pharmaceutical compositions of the invention can additionally contain suitable buffering agents, including, for example, acetic acid in a salt, citric acid in a salt, boric acid in a salt, and phosphoric acid in a salt. The compositions can also optionally contain suitable preservatives, such as benzalkonium chloride, chlorobutanol, parabens, and thimerosal. Pharmaceutical compositions of the invention can be presented in unit dosage form and can be prepared by any suitable method, many of which are well known in the art of pharmacy. Such methods include the step of bringing the antibody of the invention into association with a carrier that constitutes one or more accessory ingredients. In general, the composition is prepared by uniformly and intimately bringing the active agent into association with a liquid carrier, a finely divided solid carrier, or both, and then, if necessary, shaping the product.

A composition suitable for parenteral administration conveniently comprises a sterile aqueous preparation of the inventive composition, which preferably is isotonic with the blood of the recipient. This aqueous preparation can be formulated according to known methods using suitable dispersing or wetting agents and suspending agents. The sterile injectable preparation also can be a sterile injectable solution or suspension in a non-toxic parenterally-acceptable diluent or solvent, for example, as a solution in 1,3-butane diol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed, such as synthetic mono- or di-glycerides. In addition, fatty acids such as oleic acid can be used in the preparation of injectables. Carrier formulations suitable for oral, subcutaneous, intravenous, intramuscular, etc. administrations can be found in Remington's Pharmaceutical Sciences, Mack Publishing Co., Easton, PA.

Preparation of pharmaceutical compositions of the invention and their various routes of administration can be carried out in accordance with methods well known in the art. See, e.g., Remington: The Science and Practice of Pharmacy, Mack Publishing Co., 20th ed., 2000; and Sustained and Controlled Release Drug Delivery Systems, J. R. Robinson, ed., Marcel Dekker, Inc., New York, 1978. The delivery systems useful in the context of the invention include time-released, delayed release, and sustained release delivery systems such that the delivery of the inventive composition occurs prior to, and with sufficient time to cause, sensitization of the site to be treated. The inventive composition can be used in conjunction with other therapeutic agents or therapies. Such systems can avoid repeated administrations of the inventive composition, thereby increasing convenience to the subject and the physician, and may be particularly suitable for certain compositions of the invention.

Many types of release delivery systems are available and known to those of ordinary skill in the art. Suitable release delivery systems include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and triglycerides; hydrogel release systems; sylastic systems; peptide based systems; wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775, 4,667,014, 4,748,034, and 5,239,660 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

### Examples

The following examples are provided to further illustrate the invention but not to limit its scope. Other variants of the invention will be readily apparent to one of ordinary skill in the art and are encompassed by the appended claims.

### Example 1. Design, Characterization and Validation of a CovCAR-T

As a prelude to using DNA-encoded libraries (DELs) to select tumor-binding molecules, we used the folate receptor (FR) and prostate-specific membrane antigen (PSMA) for proof-of-concept studies because molecules that bind to these two targets were already known.

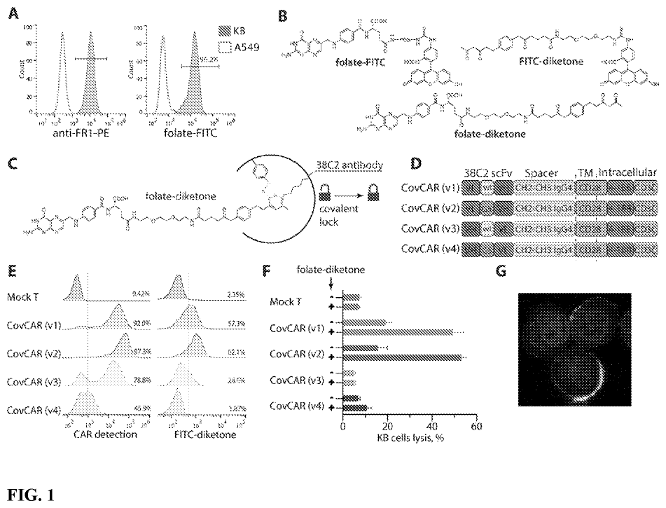

The folate receptor is one of the most widely studied cancer markers, and we used the folate receptor-positive KB cell line cancer model for our initial proof-of-concept studies. The KB cell folate receptor expression level was verified by both antibody and folate-PEG-FITC dye staining (FIG. 1-A). We envisioned that small molecule linkers would be able to tether the catalytic CAR-T cells to cancer cells and initiate cytolysis. To validate our design, we synthesized another bifunctional molecule that has diketone on one end and folate on the other end, joined by a short PEG chain (FIG. 1-B). The 1,3-diketone reacts with a lysine residue that is deeply buried in the hydrophobic environment of the antibody binding pocket. As such, this lysine is not protonated at physiological pH because a positive charge is disfavored in a hydrophobic environment and thus remains nucleophilic (pKa perturbed by 3 orders of magnitude). The nucleophilic attack of the 1,3-diketone by this unprotonated lysine results in the formation of a covalent enamineone (FIG. 1-C) (Wirsching P et. al., Science 270:1175-82, 1995).

The original covalent bond-forming catalytic antibody (clone 38C2) was an IgG. To re-purpose it for the construction of CAR-Ts, we first converted the antibody to different single chain fragment variable (scFv) configurations and screened for the best format (FIG. 1-D). We found that the order of heavy and light chains, as well as the linker sequence, had significant impact on the CAR expression level on the cell surface (FIG. 1-E). To determine if the scFv format still preserved the ability to form covalent bonds, we synthesized a FITC-diketone molecule (FIG. 1-B) and examined its ability to bind to the 38C2 antibody on the cell surface. The staining and analysis by flow cytometry, together with the cytotoxicity analysis against KB cells, showed that the CovCAR (v2) configuration gave the strongest signal (FIG. 1-F). Therefore, we chose CovCAR (v2) for future experiments, since it allowed the highest functional surface copy number, surface covalent modification, and cytotoxicity against cancer cells (FIG. 1-E, F and G).

### Example 2. Activation of CovCAR-T In Vitro and In Vivo Activity

This Example descries activation by small molecule switch of CovCAR-T in vitro and in vivo activity on folate receptor-positive cancer.

The human folate receptor (FR) exists in multiple isoforms, but only FRa and FRb are cell-surface glycoproteins with potential utility for cancer therapy. FRa is mostly expressed on epithelial cancer types, including ovarian, cervical, renal cell carcinoma, non-small cell lung cancer, and triple-negative breast cancer Cheung A, et al., Oncotarget. 7:52553-74, 2016; and Elnakat et al., Adv Drug Deliv Rev. 56:1055-1237, 2004. FRb is expressed on a few hematologic malignancies, including AML Lynn R C, et. al., Blood. 125:3466-76, 2015, and on a subset of tumor-associated macrophages Kurahara et. al., Pancreas. 42:155-9, 2013. CAR-T therapies directed against FRa and FRb have been explored using different direct tumor recognition CAR constructs. See, e.g., Song et al., Cancer Res. 71:4617-27, 2011; Xu et al., Oncotarget. 7:82354-68, 2016; Song et al., J Hematol Oncol. 9:56, 2016; Kim et. al., PLOS One. 13: e0198347,2018. The main challenge for all of them has been to focus the T cell sensitivity to FR-overexpressing cells only, since FR is also expressed at low levels on normal epithelium. In this regard the small drug conjugate mediator, which bridges the tumor and CAR-T cells, allows one to adjust the CovCAR-T cell sensitivity for the tumor antigen density.

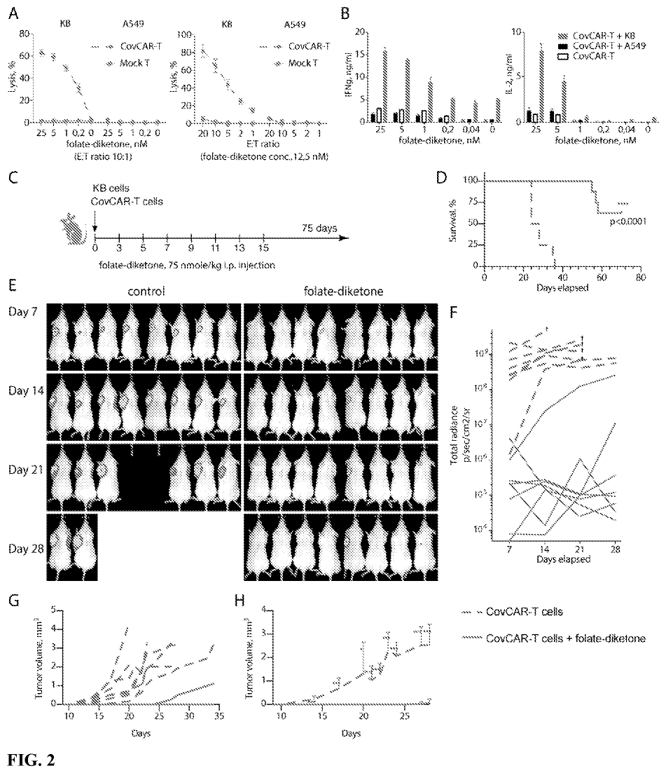

When incubated with target carcinoma FR-expressing KB cells in the presence of folate-diketone, covalent CAR-Ts demonstrated well-controlled cytotoxicity (FIG. 2-A), while folate receptor negative cells (A549) were not affected. Later, careful titration of the folate-diketone concentration and cell ratio (E: T ratio) showed CovCAR-Ts can efficiently lyse the target KB cells and release T cell activation marker cytokines (FIG. 2-A and B) in the presence of the adapter molecule, with an EC50 of 1 nM.

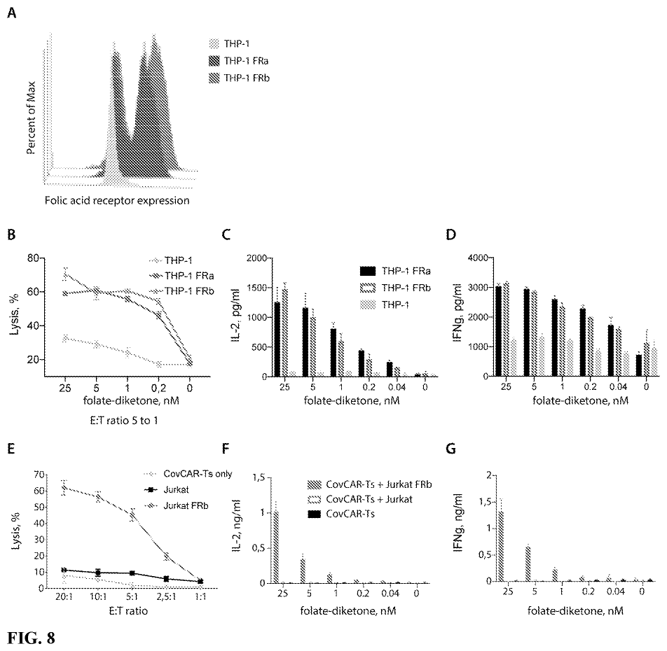

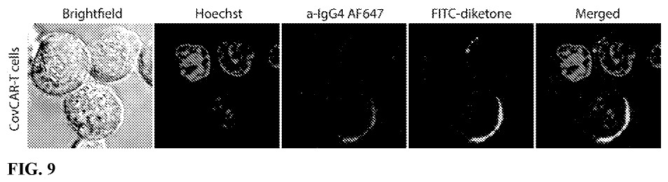

To represent pediatric malignancies, we utilized AML THP-1-expressing FRa and FRb cells. Co-incubation of the THP-1 FRa- or FRb-expressing cells with CovCAR-Ts demonstrated dose dependent cytotoxicity and cytokine release (FIG. 7).

To validate the small molecule-regulated CovCAR-T activity in vivo, we injected mice subcutaneously with KB tumor cells together with CovCAR-Ts, with or without daily folate-diketone administration for the following two weeks (FIG. 2-C). The mice were monitored until 75 days after tumor inoculation. The control group (CovCAR-Ts alone without folate-diketone) mice developed normal-sized subcutaneous tumors (FIG. 2-D and E) and had a shortened life span. In contrast, mice treated with CovCAR-Ts plus folate-diketone showed significantly improved survival and greatly reduced tumor volume (FIG. 2-E, F, G and H). This indicates the bifunctional small molecule switch is able to regulate CovCAR-T in vivo activity and suppress folate receptor-positive tumor growth.

### Example 3. Activation of CovCAR-T In Vitro and In Vivo Activity Against Prostate Cancer

This Example descries activation by small molecule switch of CovCAR-T in vitro and in vivo activity on prostate cancer.

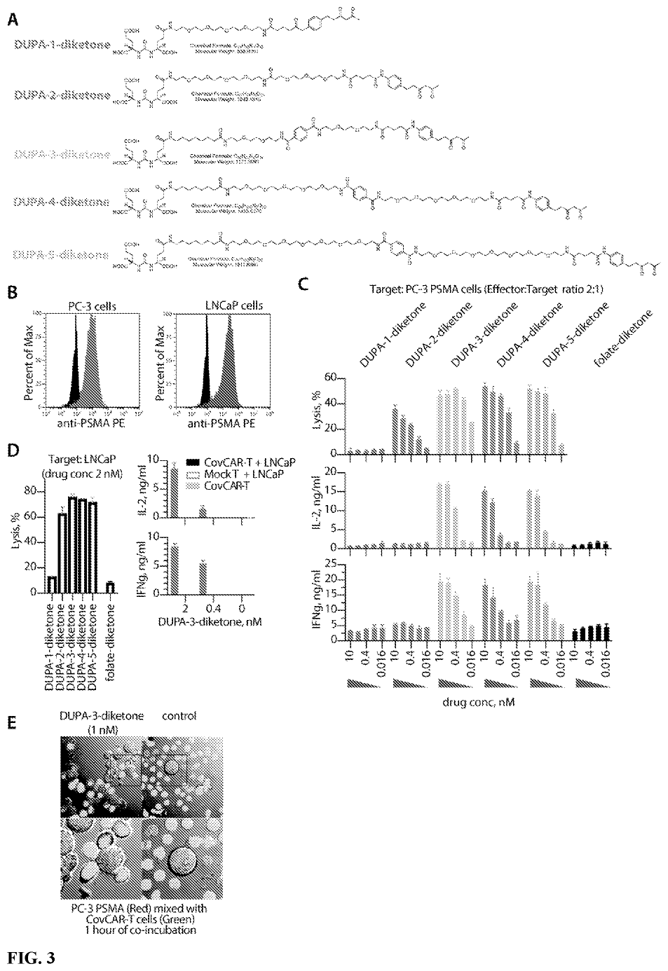

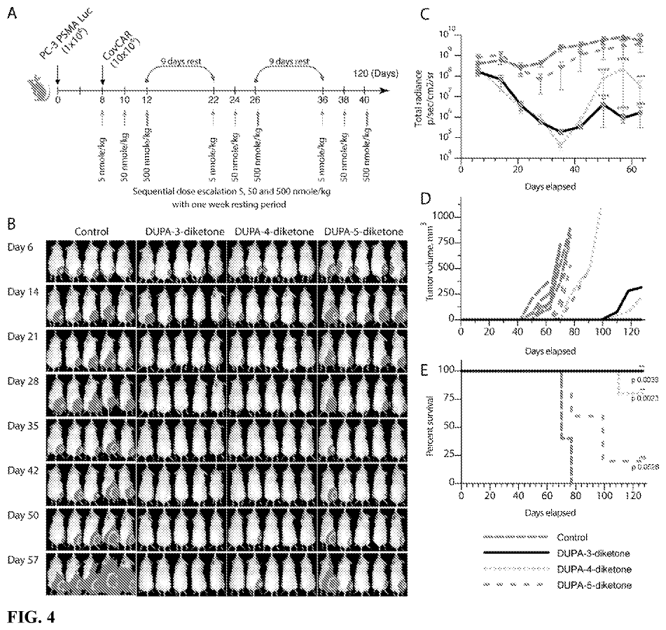

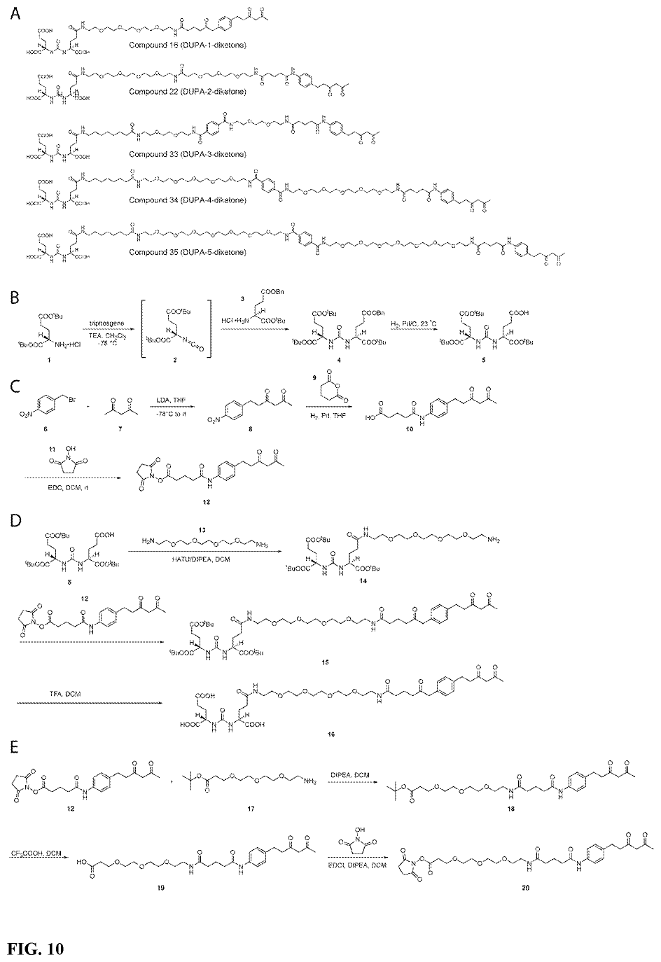

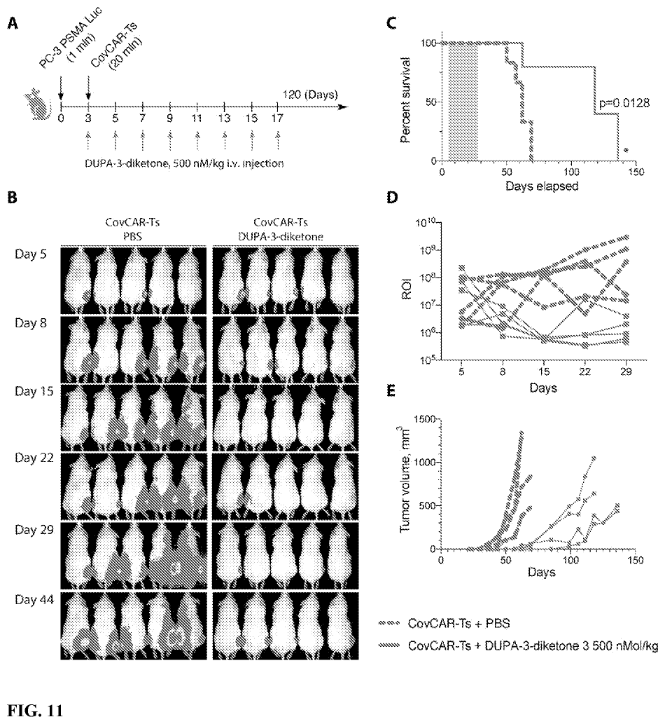

Prostate cancer is the most common malignant cancer and the second leading cause of cancer-related death among men in the United States. PSMA is a type II membrane protein and has been exploited as an ideal target for diagnosis and treatment of prostate cancer. The already known DUPA-targeting moiety belongs to a class of glutamate ureas and can bind PSMA selectively with nM affinity. Therefore, for further proof-of-concept studies using prostate cancer we designed a panel of the small adapter molecules based on DUPA functionalities conjugated to 1,3-diketone. Because spacer length may impact efficacy of targeted drug conjugates with DUPA (Peng et al., J Drug Target 21:968-80, 2013), we synthesized five DUPA-diketone conjugates with different linkers (FIG. 3-A, FIG. 10). We selected two PSMA-expressing cell lines as target cells, PC-3 PSMA+ and LNCaP (FIG. 3-B). We compared CovCAR-T-mediated cytotoxicity and proinflammatory cytokine release against PSMA-expressing PC-3 PSMA+ and LNCaP cell lines in the presence of different concentrations of each of the five DUPA-diketone conjugates (FIG. 3-C). We found a linear correlation between DUPA-diketone spacer length and CAR-T functional activity. Compounds DUPA-3-diketone, DUPA-4-diketone and DUPA-5-diketone provided the best tumor lysis and cytokine release, with effective concentration ranges from 10 nM to 20 μM. While compound DUPA-1-diketone was non-active, the DUPA-2-diketone conjugate showed a low level of lysis and cytokine release. As a result, we not only optimized the DUPA-diketone conjugate structure, but also demonstrated that the therapeutic effect of CovCAR-Ts may be modulated as a function of the linker length between the adapter molecules and the T cells. Similar results were obtained with the LNCaP cell line (FIG. 3-D). The illustrative confocal imaging of CovCAR-Ts attacking the prostate cancer cells only in presence of the DUPA-3-diketone confirms programmable cytolysis of tumors by the modified lymphocytes (FIG. 3-E).

Unlike the conventional fixed CAR design, the CovCAR modular design allows targeting of multiple antigens without further genetic manipulations of a patient's immune cells. To test the ability of the CovCAR system we prepared a mixture of the PC-3, PC-3 PSMA and PC-3 FRa cells. The CovCAR-Ts were co-incubated with multiple target cell mixtures (E: T ratio 1:2) in presence of the folate-diketone and DUPA-3-diketone in different concentrations (0, 0.2 or 1 nM). After eight hours of co-incubation, we counted the number of live PC-3, PC-3 PSMA and PC-3 FRa cells. As expected, the addition of diketone conjugates targeting either FRa, PSMA, or both led to tunable killing efficiency without affecting the “normal” PC-3 cells, illustrating the potential of programming the CovCAR-Ts to combat heterogeneous tumors or antigen escape.

After confirmation of the in vitro activity, we analyzed the therapeutic efficacy of the three best DUPA-3-diketone, DUPA-4-diketone and DUPA-5-diketone compounds in vivo. SCID mice were subcutaneously transplanted with 1 million luciferase-expressing PC-3 PSMA+ cells. Eight days after tumor implantation animals were intravenously injected (i.v.) with 10 million CovCAR-Ts. Six hours after CovCAR-T injection we initiated therapy with varying doses of the DUPA-diketone conjugates (FIG. 4). The dose escalation therapeutic protocol consists of cycles of small molecule injections at doses 5/50/500 nmol per kg every other day as shown (FIG. 4-A). The dose escalation cycles are separated by resting periods (9-12 days). This protocol of drug administration was found not only to protect against CRS (Lu et al., Front Oncol. 9:151, 2019), but also to improve CAR-T persistence and to delay exhaustion of the modified T cells (Weber, et. al., Science. 372: eaba1786, 2021).

In vivo imaging of animals from the control and therapeutic groups demonstrated the outstanding therapeutic potential of our CovCAR-T/DUPA-diketone conjugate methodology against prostate cancer in vivo (FIG. 4-B and C). Animals treated with DUPA-3-diketone and DUPA-4-diketone conjugates exhibited complete tumor elimination, and high survival rates after the first cycle of drug administration (FIG. 4-D and E). Surprisingly, the DUPA-5-diketone compound with the longest spacer showed slower tumor elimination, incomplete suppression of tumor growth, and minimal survival benefit, again indicating the importance of controlling the space between T cells and their tumor target. This ability to adjust T cell efficacy as a function of linker length provides another degree of control in the clinical setting.

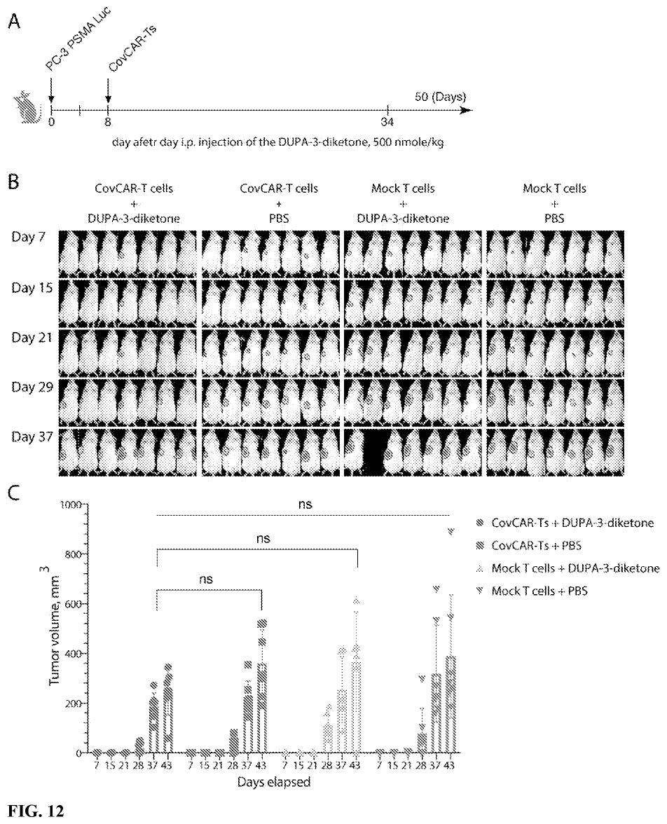

We also studied different strategies of drug administration, such as intravenous injection of 500 nMol/kg of DUPA-3-diketone every second day. Animals treated with i.v. injections of 500 nMol/kg of DUPA-3-diketone exhibited tumor elimination and extended survival (FIG. 11), although this regimen was not as effective as the dose escalation protocol. Drug delivery by i.p. injections did not demonstrate any therapeutic effect (FIG. 12).

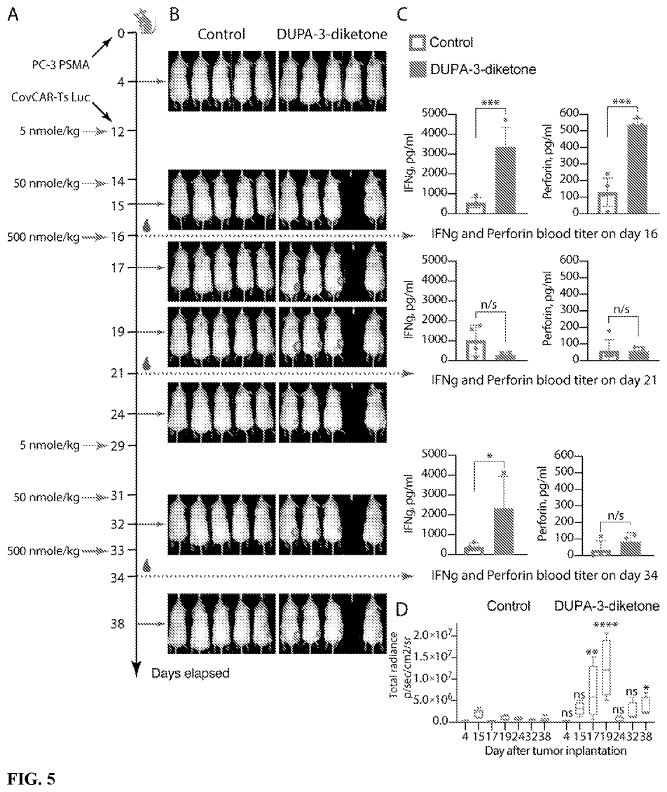

Next, we confirmed that diketone-conjugates allow control of the efficacy and persistence of CovCAR-Ts during the therapy. For tracking of the therapeutic T cells, we designed CAR-T cells expressing both the CovCAR and luciferase as a marker (CovCAR-Ts Luc). SCID mice were subcutaneously transplanted with 1 million PC-3 PSMA+ cells without luciferase. IVIS on day four demonstrates that tumor-bearing animals do not have luciferase signals. Twelve days after tumor implantation animals were injected with 10 million CovCAR-Ts Luc. Six hours after CovCAR-T injection we initiated the aforementioned DUPA-3-diketone dose escalation therapeutic protocol (i.e., 5 nmol/kg on day 12, 50 nmol/kg on day 14, 500 nmol/kg on day 16, 5 nmol/kg on day 29, 50 nmol/kg on day 31, 500 nmol/kg on day 33) (FIG. 5-A).

The bioluminescent tracking of luciferase CovCAR-Ts on days 15, 17, 19, 24 and 31 shows that in the DUPA-3-diketone treated group CovCAR-T cells are located only at the site of tumors (FIG. 5-B). This observation confirms that, as intended, CAR-Ts localize uniquely to the tumor. The CovCAR-T propagation is strongly dependent on DUPA-3-diketone injections. CovCAR-Ts start to expand in a middle of first cycle of therapy as shown on days 15, 17 and 19. After drug administration was suspended, the CovCAR-Ts disappeared on day 24. But after the second round in the dose escalation protocol was initiated, they expanded again in the tumor area (FIG. 5-B and D). Serum IFNg and Perofin levels measured on days 16, 21 and 34 also correlated with observed CAR-T expansion and DUPA-3-diketone injections (FIG. 5-C). In summary, this data confirms the ability of our protocol to regulate CovCAR-T therapy in vivo.

### Example 4. Selection of the Organic Adapters from DNA-Encoded Libraries

This Example descries selection of the organic adapters from DNA-encoded libraries for multiplexed and switchable control over CovCAR-Ts.

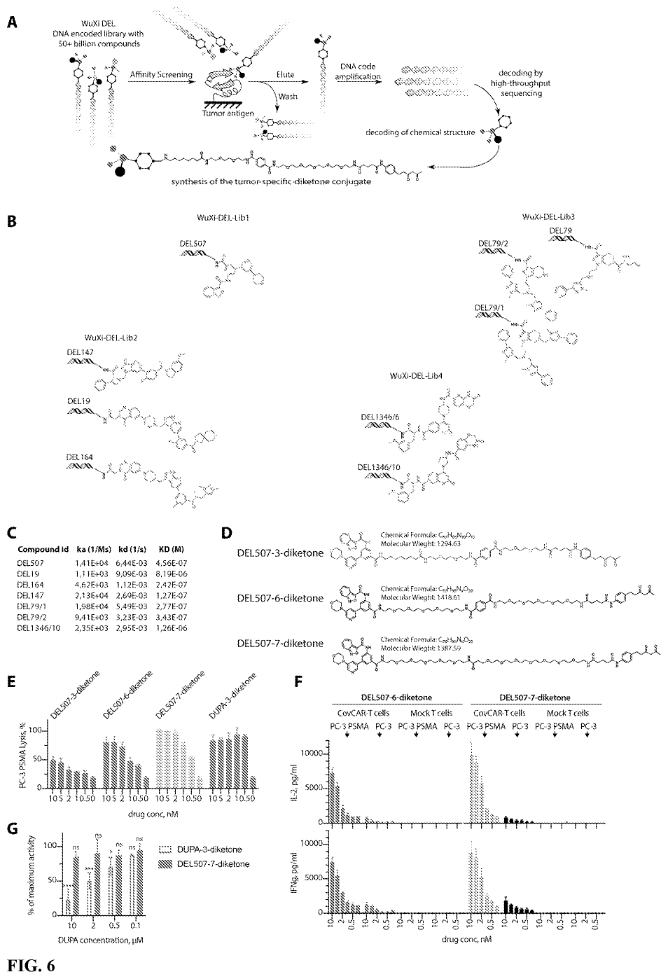

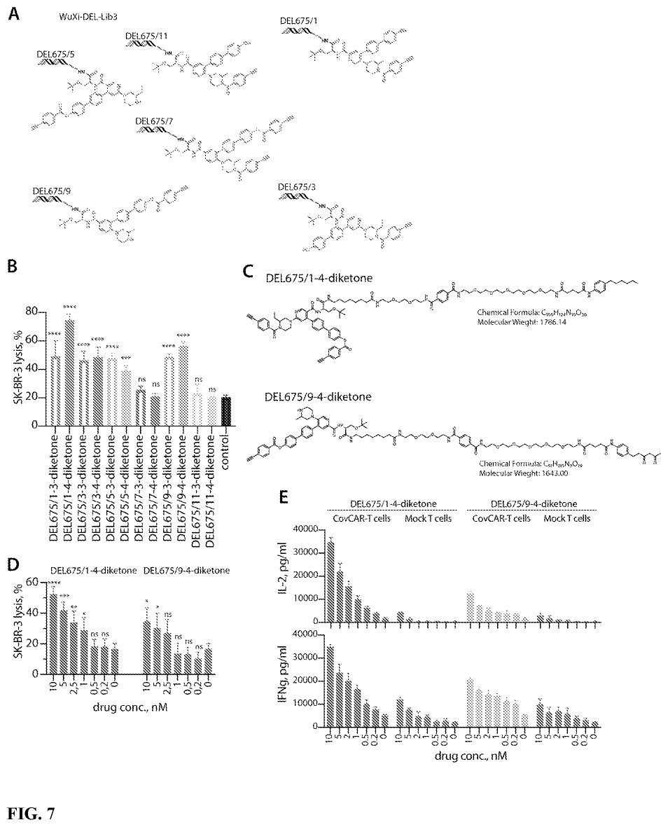

The above proof-of-concept studies demonstrated that CovCAR-Ts could work, so long as one pays attention to certain details such as linker length, but the problem remains that, as with antibodies, there are only a limited number of compounds that selectively bind to tumor cell surfaces. Since a central idea in this study is that this problem can be overcome by using the power of DELs, it was necessary to show that new molecules could be selected from such libraries. After validation of the switchable control over CovCAR-Ts by the small-drug conjugates folate-diketone and DUPA-diketone, we used a DEL to identify novel compounds that selectively bind to the cell surface of the PSMA- and HER2-expressing cancers (FIGS. 6 and 7).

Recombinant human HER2 protein was obtained from SinoBiological (Cat: 10004-H08H). The extracellular domain (Met 1-Thr 652) of human ErbB2 (NP_004439.2) with a polyhistidine tag at the C-terminus was expressed in HEK293 Cells. Recombinant human PSMA protein was obtained from SinoBiological (Cat: 15877-H07H). The extracellular domain (Lys44-Ala750) of human FOLH1 (NP_004467.1) with a polyhistidine tag at the C-terminus was expressed in HEK293 Cells. The affinity selection followed the general protocol as previously described. For polyhistidine-tagged proteins, each affinity selection used 20 L HisPur Ni-NTA magnetic beads (Thermo Fisher Scientific 88832). Beads were equilibrated 3 times with 200 μL selection buffer (1× PBS, 0.05% Tween-20, 10 mM imidazole, 0.1 mg/mL sheared salmon sperm [Invitrogen AM9680]). Proteins were immobilized on HisPur Ni-NTA magnetic beads for 0.5 h at room temperature. To exclude the background signal caused by beads, a negative control group was performed under the same conditions but without adding target protein. After immobilization, the beads, protein and DEL were incubated in 100 μL of selection buffer for 1 h at room temperature, washed three times with 200 μL of selection buffer, then eluted with 50 μL of selection buffer at 95° C. for 10 min on a Thermomixer (Eppendorf) with shaking at 800 rpm. After elution, 1 μL eluted sample was used for qPCR quantification.

Several rounds of affinity selection were performed using the same procedure. At each iteration the eluted DEL was used as the input for the next round of selection with fresh immobilized 5 μg target protein. After three-rounds of affinity selection, the samples collected from each round were quantified on QuantStudio 7 Flex Real-Time PCR System (Thermo Fisher Scientific). Samples from the final round of affinity selection were processed for PCR. The PCR cycles were determined by the qPCR quantification. Then the PCR products were purified, sequenced and analyzed as described.

As a result, we identified seven DEL compounds specific to the PSMA (FIG. 6-B). All selected organic molecules were synthesized as a spacer-diketone conjugate. The SPR measurement showed that all DEL-diketone conjugates specifically bind to their target with affinities in a nM range (FIG. 6-C).

Next, we analyzed in vitro activity of the PSMA-specific DEL-diketone conjugate (id 10037-10049-5-401-507; DEL507-diketone). Addition of the DEL507-diketone to the CovCAR-Ts and PC-3 PSMA+ cells mediates cytotoxicity and proinflammatory cytokine release in a dose-dependent manner. We found a linear correlation between the DEL507-diketone concentration and CovCAR-T functional activity, with an effective range between 10 nM to 250 μM (FIG. 6-E, G and F).

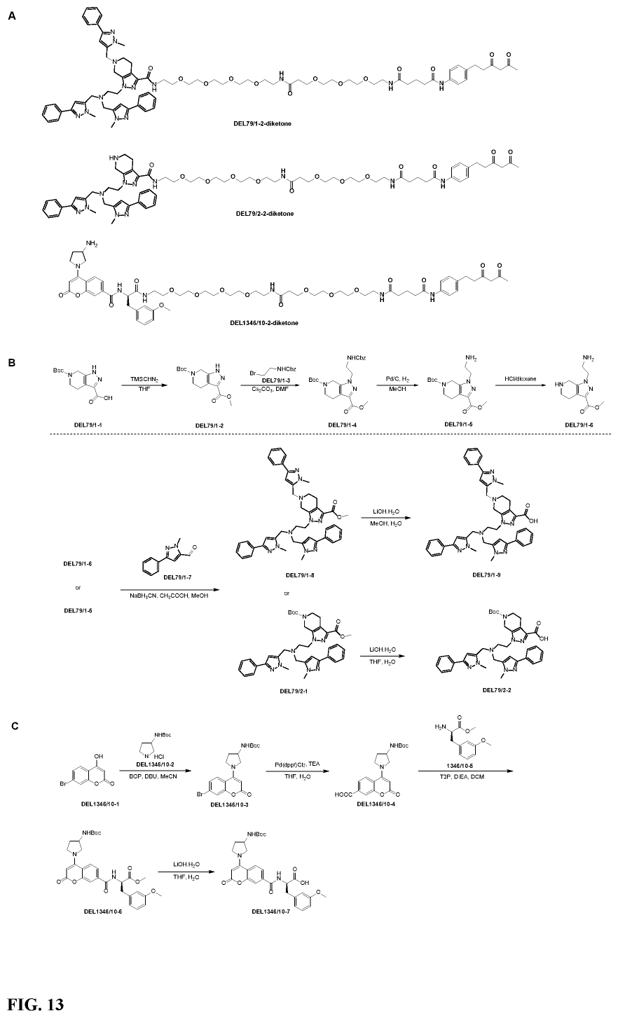

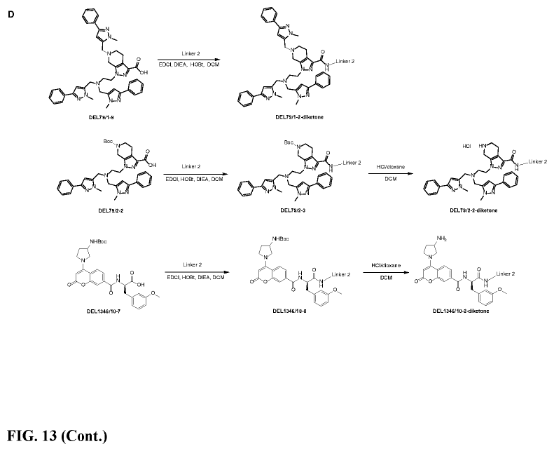

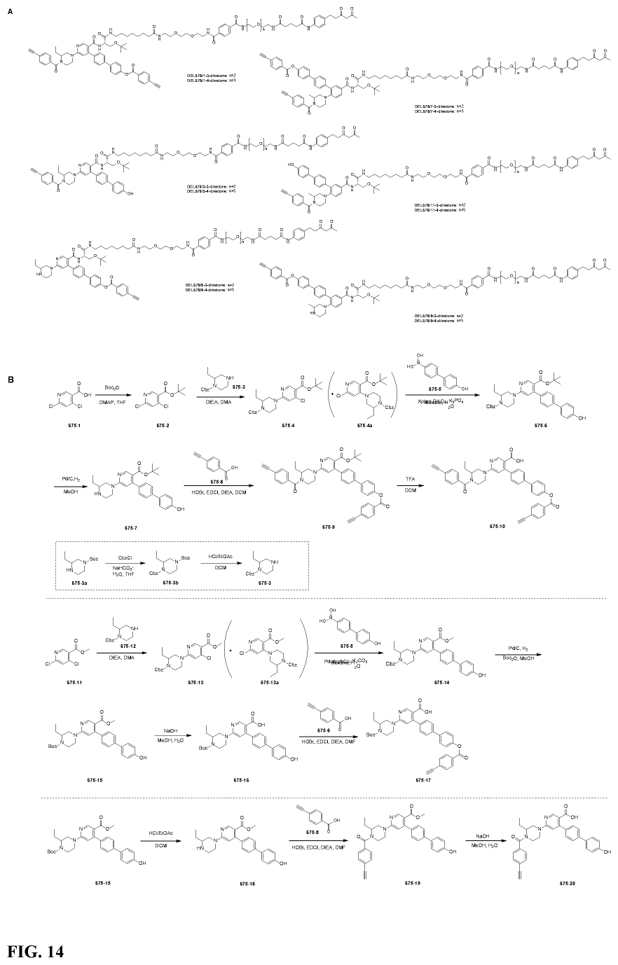

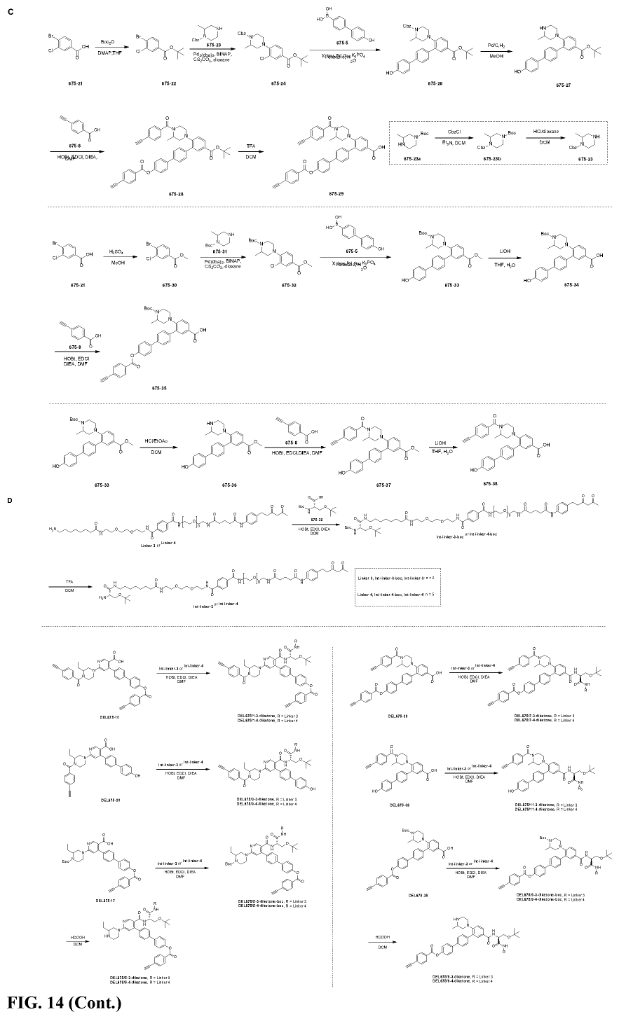

Next, we identified six DEL compounds specific to the HER2 receptor. Decoding the selected hits allowed identification of the HER2-specific molecules DEL675/1 and DEL675/3 together with potentially functional intermediates and byproducts DEL675/5, DEL675/7, DEL675/9, and DEL675/11 (FIG. 7-A, FIG. 13, and FIG. 14). All of the identified structures were synthesized as spacer-diketone conjugates with two types of linkers and then analyzed for in vitro activity on CovCAR-Ts and HER2 positive SK-BR-3 cells (FIG. 7-B). Addition of the DEL675/1-4-diketone and DEL675/9-4-diketone conjugates (FIG. 7-C) to the CovCAR-Ts and SK-BR-3 cells mediated cytotoxicity and proinflammatory cytokine release in a dose-dependent manner (FIG. 7-D and E).

The exemplary PSMA specific DEL compounds identified from the studies are shown in FIG. 6. These include, e.g., compounds DEL507, DEL147, DEL19, DEL164, DEL79, DEL79/2, DEL79/1, DEL1346/6, and DEL 1346/10. The exemplary HER2 specific DEL compounds identified from the studies are shown in FIG. 7. These include, e.g., compounds DEL675/1, DEL675/3, DEL675/5, DEL675/9, and DEL675/11.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

We claim:

1. A chimeric antigen receptor T (CAR-T) cell composition for targeting a tumor cell, comprising (a) a CAR-T cell comprising in the extracellular domain of its CAR a catalytic antibody, and (b) an adapter compound comprising a substrate moiety of the catalytic antibody that is linked to a targeting moiety that specifically recognizes a surface molecule of the tumor cell.

2. The composition of claim 1, wherein the catalytic antibody is a single chain fragment variable (scFv) molecule.

3. The composition of claim 1, wherein the catalytic antibody possesses the activity of catalyzing formation of a covalent bond between the antibody and the substrate moiety.

4. The composition of claim 3, wherein the covalent bond is formed between the substrate moiety and a reactive amino acid residue of the catalytic antibody.

5. The composition of claim 3, wherein the catalytic antibody is catalytic antibody 38C2 or variant thereof.

6. The composition of claim 5, wherein the catalytic antibody is humanized antibody 38C2.

7. The composition of claim 5, wherein the variant is a scFv of h38C2.

8. The composition of claim 7, wherein the scFv comprises the light chain variable region (VL) sequence of antibody 38C2 that is linked at its C-terminus by a Gly-Ser linker to the N-terminus of the heavy chain variable region (VH) sequence of antibody 38C2.

9. The composition of claim 3, wherein the substrate moiety comprises a small molecule.

10. The composition of claim 3, wherein the substrate moiety comprises 1,3-diketone or β-lactam.

11. The composition of claim 1, wherein the targeting moiety is identified via screening a DNA-encoded compound library for specific binding to the tumor cell surface molecule.

12. The composition of claim 1, wherein the targeting moiety targets PSMA.

13. The composition of claim 12, wherein the targeting moiety comprises compound DEL507, DEL147, DEL19, DEL164, DEL79, DEL79/2, DEL79/1, DEL 1346/6 or DEL 1346/10.

14. The composition of claim 1, wherein the targeting moiety targets HER2.

15. The composition of claim 14, wherein the targeting moiety comprises compound DEL675/1, DEL675/3, DEL675/5, DEL675/9 or DEL675/11.

16. A method for targeting a CAR-T cell to a tumor cell present in a population of heterogeneous cells, comprising contacting the population of cells with a CAR-T composition comprising (a) a CAR-T cell comprising in the extracellular domain of its CAR a catalytic antibody, and (b) an adapter compound comprising a substrate moiety of the catalytic antibody that is linked to a targeting moiety that specifically recognizes a surface molecule of the target tumor cell; thereby targeting the CAR-T cell to the tumor cell.

17. The method of claim 16, wherein the catalytic antibody catalyzes formation of a covalent bond between the antibody and the substrate moiety.

18. The method of claim 17, wherein the covalent bond is formed between the substrate moiety and a reactive amino acid residue of the catalytic antibody.

19. The method of claim 17, wherein the catalytic antibody is catalytic antibody 38C2 or variant thereof.

20. The method of claim 17, wherein the catalytic antibody is humanized antibody 38C2 or an antibody fragment thereof.

21. The method of claim 20, wherein the antibody fragment is a scFv of h38C2.

22. The method of claim 17, wherein the substrate moiety comprises a small molecule.

23. The method of claim 17, wherein the substrate moiety comprises 1,3-diketone or β-lactam.

24. The method of claim 16, further comprising identifying the targeting moiety via screening a DNA-encoded compound library for specific binding to the tumor cell surface molecule.

25. The method of claim 16, wherein the tumor cell is breast cancer cell or prostate cancer cell.

26. A method for treating a cancer in a subject, comprising administering to the subject a pharmaceutical composition comprising (a) a CAR-T cell comprising in the extracellular domain of its CAR a catalytic antibody, and (b) an adapter compound comprising a substrate moiety of the catalytic antibody that is linked to a targeting moiety that specifically recognizes a surface molecule of the cancer; thereby treating the cancer in the subject.

27. The method of claim 26, wherein the catalytic antibody catalyzes formation of a covalent bond between the antibody and the substrate moiety.

28. The method of claim 27, wherein the covalent bond is formed between the substrate moiety and a reactive amino acid residue of the catalytic antibody.

29. The method of claim 27, wherein the catalytic antibody is catalytic antibody 38C2 or variant thereof.

30. The method of claim 27, wherein the catalytic antibody is humanized antibody 38C2 or an antibody fragment thereof.

31. The method of claim 30, wherein the antibody fragment is a scFv of h38C2.

32. The method of claim 27, wherein the substrate moiety comprises a small molecule.

33. The method of claim 27, wherein the substrate moiety comprises 1,3-diketone or β-lactam.

34. The method of claim 26, further comprising identifying the targeting moiety via screening a DNA-encoded compound library for specific binding to the tumor cell surface molecule.

35. The method of claim 26, wherein the cancer is breast cancer or prostate cancer.
